(19) United States
(12) Patent Application Publication
(10) Pub. No.: US 2016/0303150 A1
(43) Pub. Date: Oct. 20, 2016

(54) DEUTERATED TREHALOSE FORMULATIONS AND USES THEREOF

(71) Applicant: BioBlast Pharma Ltd., Tel Aviv (IL)

(72) Inventor: Dalia MEGIDDO, M.P. Judean Hills (IL)

(21) Appl. No.: 15/099,168

(22) Filed: Apr. 14, 2016

Related U.S. Application Data
(60) Provisional application No. 62/148,530, filed on Apr. 16, 2015.

(51) Int. Cl. A61K 31/7016; A61K 9/08; A61K 9/00

(52) U.S. Cl. CPC A61K 31/7016; A61K 9/0019; A61K 9/0053; A61K 9/08

(57) ABSTRACT

The presently invention relates to methods, formulations and kits comprising deuterated trehalose for treating myopathies, neurodegenerative disorders, or tauopathies associated abnormal protein aggregation.

### CROSS-REFERENCE TO RELATED APPLICATION

This application claims priority under 35 U.S.C. §119 to U.S. Provisional Application Ser. No. 62/148,530, filed Apr. 16, 2016, the contents of which is incorporated herein by reference in its entirety.

### TECHNOLOGICAL FIELD

The presently disclosed subject matter relates to use of deuterated trehalose in the treatment of myopathies, neurodegenerative disorders or tauopathies associated abnormal protein aggregation.

### BACKGROUND ART

References considered to be relevant as background to the presently disclosed subject matter are listed below:
[1] Brunet, G. et al., 1990 Dystrophie musculaire oculo-pharyngée. Recensement des Familles Françaises et Études Généalogiques. Rev Neurol 4:429-434.
[2] Blumen, S. C. et al., 1997 Epidemiology and inheritance of oculopharyngeal muscular dystrophy in Israel. Neuromuscul Disord 7:S38-40.

[3] Becher, M. W. et al., 2001 Occulopharyngeal Muscular Dystrophy in Hispanics New Mexicans. JAMA. 286(19):2437-40.
[4] Grewal, R. J. et al., 1999 Mutation Analysis of Oculopharyngeal Muscular Dystrophy in Hispanic American Families. Arch Neurol 56(11):1378-1381.
[5] McClellan, A. J. & Frydman, J. 2001. Molecular chaperones and the art of recognizing a lost cause. Nat Cell Biol 3:E51-E53.
[6] Winklhofer, K. F. et al., 2001. Geldanamycin restores a defective heat shock response in vivo. J Biol Chem 276:45160-45167.
[7] Tanaka, M. et al., 2004. Trehalose alleviates polyglutamine-mediated pathology in a mouse model of Huntington disease. Nat Med 10:148-154.
[8] Davies, J. E. et al., 2006. Trehalose reduces aggregate formation and delays pathology in a transgenic mouse model of oculopharengeal muscular dystrophy. Hum Mol Genet 15:23-31.
[9] WHO Food Additives at the world wide web (www).inchem.org/documents/jecfa/jecmono/v46je05.htm.
[10] Oral Trehalose Therapy to Reverse Arterial Aging in Middle-Aged and Older Adults. World wide web (www) clinicaltrials.gov/ct2/show/NCT01575288.
[11] WO 2014/181333.
[12] Berg, N. O. et al., 1963. Correlation between morphological alterations and enzyme activities in the mucosa of the small intestine. Scand J Gastroenterol 8:703-712.
[13] Hore, P. & Messer, M. 1968. Studies on disaccharidase activities in the small intestine of domestic cats and other carnivorous mammals. Comp Biochem Physiol 24:717-725.
[14] Buteau, K. C. 2009. Deuterated Drugs: Unexpectedly Nonobvious? Journal of High Technology Law 22.
[16] World wide web (www) tga.gov.au/pdf/euguide/ich822602006.pdf.
[17] Langer R 1990. New methods of drug delivery. Science 28; 249:1527-1533.
[18] Li, J. Y. et al., 2005. The Use of the R6 Transgenic Mouse Models of Huntington's Disease in Attempts to Develop Novel Therapeutic Strategies. The Journal of the American Society for Experimental NeuroTherapeutics 2: 447-464.

Acknowledgement of the above references herein is not to be inferred as meaning that these are in any way relevant to the patentability of the presently disclosed subject matter.

### BACKGROUND

Several groups of diseases resulting from trinucleotide repeat mutations are known. These diseases are characterized by abnormal stretches of amino acids in specific proteins encoded by a mutated gene. The mutant protein aggregates in cells causing typical citotoxic cellular inclusion bodies.

Disorders identified as protein codon reiteration disorders contain expansions of a homopolymeric stretch of amino acids, specifically polyglutamine (poly Q) or polyalanine (poly A). At least eight neurodegenerative disorders have been associated with polyglutamine expansions, including Huntington's disease (HD), spinal and bulbar muscular atrophy (SBMA), dentatorubral and pallidoluysian atrophy (DRPLA), and several forms of spinocerebellar ataxia (SCA). Polyalanine expansions are associated with several rare and severe congenital abnormalities, but also with oculopharyngeal muscular dystrophy (OPMD). Additional conditions associated with polyalanine expansions may include dystrophic disorders.

Huntington's disease (HD), associated with expansions of a homopolymeric stretch of polyglutamine (poly Q), is a neurodegenerative genetic disorder that affects muscle coordination and leads to mental decline and behavioral symptoms. Symptoms of the disease can vary between individuals and affected members of the same family, but usually progress predictably. While earliest symptoms are often subtle problems with mood or cognition, a general lack of coordination and an unsteady gait often follows, and as the disease advances, uncoordinated, jerky body movements become more apparent, along with a decline in mental abilities and behavioral symptoms. Physical symptoms can begin at any age from infancy to old age, but usually begin between 35 and 44 years of age. HD is the most common genetic cause of abnormal involuntary writhing movements called chorea.

The disease is caused by an autosomal dominant mutation in either of an individual's two copies of the Huntingtin gene, encoding the protein “huntingtin”. Expansion of a CAG (cytosine-adenine-guanine, encoding glutamine) triplet repeat stretch within the Huntingtin gene results in a different form of the protein, which gradually damages cells in the brain. A child of an affected person typically has a 50% chance of inheriting the disease.

The disease can affect both men and women. There is currently no cure for HD, and full-time care is required in the later stages of the disease. Existing pharmaceutical and non-drug treatments can relieve many of its symptoms. It is much more common in people of Western European descent than in those of Asian or African ancestry.

Accordingly, there is an urgent need for compositions and therapeutic methods for alleviating the signs and symptoms of Huntington's Disease.

Oculopharyngeal Muscular Dystrophy (OPMD), associated with expansions of a homopolymeric stretch of polyalanine (poly A), is a rare inherited myopathy characterized by ptosis, severe dysphagia and proximal limb weakness. Its estimated prevalence is 1:100,000 and the largest clusters reported were in families of French-Canadians origin in Canada and in the US (prevalence 1:1000), Bukhara Jews in Israel (prevalence 1:600) and Hispanics in New Mexico, Arizona Colorado and California [1-4]. OPMD is inherited, in most cases, as an autosomal dominant trait with complete penetrance. The disease is equally prevalent among both genders. The gene associated with the disease encodes the binding protein nuclear 1 protein (PABPN 1), a nuclear protein involved in pre-mRNA polyadenylation, transcription regulation, and mRNA nucleogytoplasmic transport. The mutation causing OPMD results in production of an abnormal poly (A) PABPN 1.

The disease is most often diagnosed in the fifth-sixth decades of life and progresses throughout the patient's life. By the age of 70, the majority of patients suffer from all or some of the following symptoms: severe dysphagia, ptosis, tongue atrophy and weakness, lower and upper limb proximal weakness, dysphonia, limitation in upward gaze and facial muscle weakness. As ptosis becomes more pronounced patients adapt the “astronomer posture” tilting of the head and upward gaze—further aggravating the dysphagia. The dysphagia starts with difficulty in swallowing solid food and progresses to liquids as well. As the dysphagia becomes more severe, patients become malnourished, cachectic, dehydrated and suffer from repeated aspiration pneumonia. OPMD does not seem to shorten life expectancy but is associated with severe debilitation and reduced quality of life.

There is no medical treatment or potential cure for OPMD. Current therapeutic strategies are confined to surgical interventions aimed at alleviating ptosis. Repeated cricopharyngeal dilatations are frequently used to relieve dysphagia. Myotomy of the upper esophageal sphincter muscles has also been employed. These procedures may provide only temporary relief and do not affect the progression of the disease that eventually leads to severe difficulty in swallowing, recurrent aspiration with increasing risk of aspiration pneumonia and severe weight loss which are the most common causes of mortality in OPMD patients.

Accordingly, there is an urgent need for compositions and therapeutic methods for alleviating the signs and symptom s of oculopharyngeal muscular dystrophy.

One of the new therapeutic strategies of diseases associated with abnormal protein aggregation is to enhance the native cellular defense mechanisms against misfolded and aggregated proteins, for example using molecular chaperones which facilitate normal folding and refolding of abnormal conformations back to the native state [5]. Drugs such as geldanamycin can modulate and enhance chaperone levels [6]. Geldanamycin, however, has substantial toxicity and does not penetrate well the blood-brain barrier. In addition, oral administration of the disaccharide trehalose, a disaccharide known for its protein stabilizing effect, has been shown in HD mouse model to reduce polyglutamine aggregates, improve motor dysfunction and extend survival [7]. More recently, these findings have been reproduced upon oral administration of trehalose in a mouse model of OPMD [8].

Trehalose, a glucose disaccharide (α-G-glucopyranosyl α-D-glucopyranoside), is found in many plants, fungi, bacteria, insects and other invertebrates. Due to its unique physical and biochemical properties demonstrated in its ability to sustain and preserve a wide array of biological molecules, trehalose has found its use in several food and cosmetic products and most notably in therapeutic products. Trehalose is an approved ingredient in all major markets designated as GRAS food ingredient by the FDA [9].

Currently a clinical study is being conducted in the US which examines the effect of large amounts of orally-administered trehalose in the prevention of arterial aging. In this study, the researchers compare the effect of trehalose or maltose or a combination of the two drugs on a bio marker associated with arterial aging [10].

Applicant's co-pending WO 2014/181333 [11] disclosed a method of treatment of a disease associated with abnormal protein aggregation comprising parenterally administering pharmaceutical formulations comprising trehalose.

### GENERAL DESCRIPTION

By one of its aspects, the present disclosure provides a method for treating or alleviating a disease associated with abnormal protein aggregation and/or inclusion bodies formation in myocytes, neurons and other cells or extracellular compartments or at least one symptom associated therewith, in a human subject in need thereof comprising administering to said subject a therapeutically effective amount of deuterated trehalose or a pharmaceutical formulation comprising a therapeutically effective amount of deuterated trehalose or a mixture of several deuterated trehaloses, optionally together with non-deuterated trehalose, wherein in said deuterated trehalose at least one hydrogen atom attached to a carbon atom is replaced by a deuterium atom.

In the above and other embodiments the deuterated trehalose according to the present disclosure is α,α-deuterated trehalose.

In the above and other embodiments the deuterated trehalose according to the present disclosure is selected from α,α-[1,1′-²H₂]trehalose having a structure according to Formula I

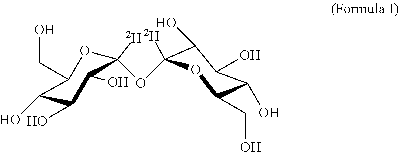

α,β-[1,1′-²H₂]trehalose having a structure according to Formula II

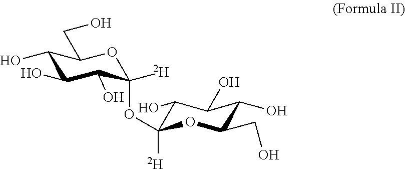

β,β-[1,1′-²H₂]trehalose having a structure according to formula III

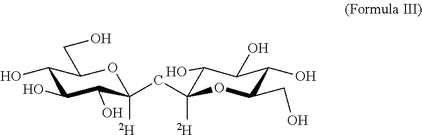

α,α-[UL-²H₁₄]trehalose having a structure according to formula IV

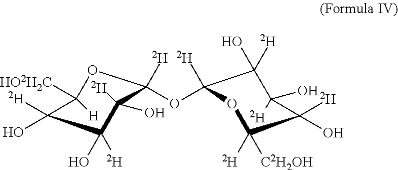

α,β-[UL-²H₁₄]trehalose, having a structure according to formula V

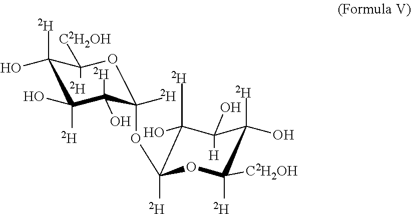

β,β-[UL-²H₁₄]trehalose having a structure according to formula VI

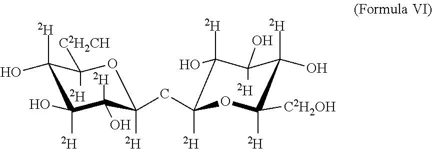

a deuterated trehalose molecule having a structure according to Formula VII

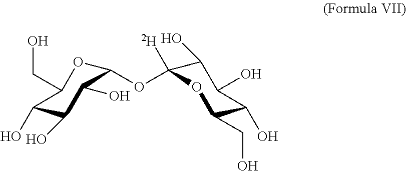

a deuterated trehalose having a structure according to Formula VIII

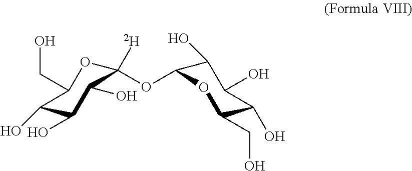

a deuterated trehalose having a structure according to Formula IX

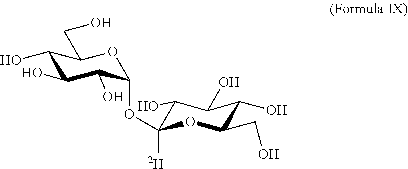

a deuterated trehalose having a structure according to Formula X

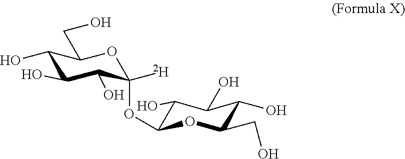

a deuterated trehalose having a structure according to Formula XI

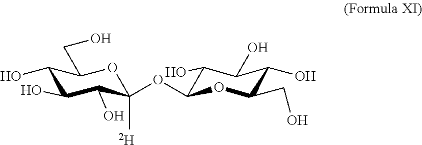

and a deuterated trehalose having a structure according to Formula XII

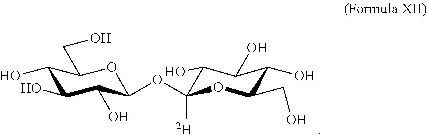

In all embodiments the disease according to the present disclosure is any one of a neurodegenerative disorder, poly-alanine aggregation disorder, poly-glutamine aggregation disorder, a protein codon reiteration disorder, a myopathy and a tauopathy.

In the above and other embodiments the disease according to the present disclosure is any one of Huntington's disease, oculopharengeal muscular dystrophy (OPMD), spinocerebellar ataxias (SCA), Friedreich's ataxia, spinal and bulbar muscular atrophy (SBMA), Parkinson's disease, Alzheimer's disease and amyotrophic lateral sclerosis (ALS), dentatorubral-pallidoluysian atrophy (DRPLA), Pick's disease, Corticobasal degeneration (CBD), Progressive supranuclear palsy (PSP) and Frontotemporal dementia and parkinsonism linked to chromosome 17 (FIDP-17).

In some embodiments the method according to the present disclosure is wherein the deuterated trehalose or mixture of several deuterated trehaloses, optionally together with non-deuterated trehalose or a pharmaceutical formulation comprising thereof is administered parenterally. In certain embodiments the pharmaceutical formulation as herein defined is an injectable solution for parenteral administration. The parenteral administration according to the present disclosure is any one of intravenous, intramuscular and intraperitoneal administration.

In other embodiments the method according to the present disclosure is wherein the deuterated trehalose or mixture of several deuterated trehaloses, optionally together with non-deuterated trehalose or a pharmaceutical formulation comprising thereof is administered enterally, specifically by oral administration.

In certain embodiments the pharmaceutical formulation as herein defined is an aqueous solution. In other embodiments the pharmaceutical formulation as herein defined is a solid dosage form.

In the above and other embodiments the pharmaceutical formulation according to the present disclosure comprises deuterated trehalose or a mixture of several deuterated trehaloses, optionally together with non-deuterated trehalose, as sole active ingredient, and optionally further comprises at least one pharmaceutically acceptable additive, carrier, excipient or diluent.

In some embodiments the concentration of deuterated trehalose or a mixture of several deuterated trehaloses, optionally together with non-deuterated trehalose in the formulation as herein defined is between about 0.1% (w/v) to about 50% (w/v). In other embodiments the concentration of deuterated trehalose in the formulation as herein defined is about 10% (w/v).

In further embodiments the pharmaceutical formulation according to the present disclosure comprising deuterated trehalose or a mixture of several deuterated trehaloses, optionally together with non-deuterated trehalose has an osmolality of from about 280 to about 330 mOsm/Kg.

In some embodiments the pharmaceutical formulation as herein defined comprises less than 0.74 endotoxin units per ml solution.

In the above and other embodiments the therapeutically effective amount of deuterated trehalose or a mixture of several deuterated trehaloses, optionally together with non-deuterated trehalose in accordance with the present disclosure is from about 1 gram to about 100 gram for each single injection and no more than about 1 gram/kg body weight of said subject per day.

In some embodiments the therapeutically effective amount of enterally administered deuterated trehalose or a mixture of several deuterated trehaloses, optionally together with non-deuterated trehalose is from about 1 mg to about 2 gram/Kg body weight of said subject per day.

In other embodiments the therapeutically effective amount of deuterated trehalose or a mixture of several deuterated trehaloses, optionally together with non-deuterated trehalose or pharmaceutical formulation comprising thereof as herein defined is administered chronically.

In other embodiments the herein defined therapeutically effective amount of deuterated trehalose or a mixture of several deuterated trehaloses, optionally together with non-deuterated trehalose or pharmaceutical formulation comprising thereof is administered at a frequency of between once daily to once per month.

In further specific embodiments the herein defined therapeutically effective amount of deuterated trehalose or a mixture of several deuterated trehaloses, optionally together with non-deuterated trehalose or deuterated trehalose or a mixture of several deuterated trehaloses, optionally together with non-deuterated trehalose comprised in said pharmaceutical formulation is administered once daily at from about 1 mg/kg/day to about 1 gram/kg/day of deuterated trehalose.

In some embodiments the therapeutically effective amount of deuterated trehalose or a mixture of several deuterated trehaloses, optionally together with non-deuterated trehalose or deuterated trehalose or a mixture of several deuterated trehaloses, optionally together with non-deuterated trehalose comprised in said pharmaceutical formulation in accordance with the present disclosure is administered at a single injection administration.

In the above and other embodiments the therapeutically effective amount as herein defined of deuterated trehalose or a mixture of several deuterated trehaloses, optionally together with non-deuterated trehalose or pharmaceutical formulation comprising the same is intravenously administered at a single dose of from about 5 to about 35 grams of deuterated trehalose, which may be administered once daily, once every other day, twice a week, once a week, once every two weeks, once every three weeks or once a month.

In some embodiments the therapeutically effective amount as herein defined of deuterated trehalose or deuterated trehalose or a mixture of several deuterated trehaloses, optionally together with non-deuterated trehalose comprised in said pharmaceutical formulation is administered at a single dose of 5, 8, 15, 30, 40 or 50 grams of deuterated trehalose.

In other embodiments the pharmaceutical formulation according to the present disclosure is an injectable solution and the rate of administration is such that the maximum endotoxin level is less than 5 endotoxin units per kilogram of body weight per hour.

In further embodiments administration as herein defined of said therapeutically effective amount of deuterated trehalose or a mixture of several deuterated trehaloses, optionally together with non-deuterated trehalose comprised in said pharmaceutical formulation adapted for intravenous administration is completed within from about 75 to about 120 minutes, specifically within less than 90 minutes.

In certain embodiments the administration as herein defined comprises a dosing regimen of equal doses, or gradually increasing doses, or gradually decreasing doses of deuterated trehalose or a mixture of several deuterated trehaloses, optionally together with non-deuterated trehalose or pharmaceutical formulation comprising the same.

In some embodiments the therapeutically effective amount of deuterated trehalose or a mixture of several deuterated trehaloses, optionally together with non-deuterated trehalose or pharmaceutical formulation comprising thereof as herein defined is administered periodically.

By another one of its aspects, the present disclosure provides deuterated trehalose or a mixture of several deuterated trehaloses, optionally together with non-deuterated trehalose or a pharmaceutical formulation comprising same, for use in a method for treating or alleviating a disease associated with abnormal protein aggregation and/or inclusion bodies formation in myocytes, neurons and other cells or extracellular compartments or at least one symptom associated therewith, in a human subject in need thereof, said method comprising administering to said subject a therapeutically effective amount of deuterated trehalose or a mixture of several deuterated trehaloses, optionally together with non-deuterated trehalose or a pharmaceutical formulation comprising the same, wherein in said deuterated trehalose at least one hydrogen atom attached to a carbon atom is replaced by a deuterium atom.

By yet another one of its aspects, the present disclosure provides an aqueous pharmaceutical formulation for any one of enteral or parenteral administration, comprising a therapeutically effective amount of deuterated trehalose or a mixture of several deuterated trehaloses, optionally together with non-deuterated trehalose as a sole active ingredient, wherein in said deuterated trehalose at least one hydrogen atom attached to a carbon atom is replaced by a deuterium atom, optionally further comprising at least one of pharmaceutically acceptable additive, excipient, diluent and carrier.

In some embodiments the aqueous pharmaceutical formulation according to the present disclosure is adapted for parenteral administration. In certain embodiments the aqueous pharmaceutical formulation as herein defined is adapted for intravenous, intramuscular or intraperitoneal administration.

In other embodiments the aqueous pharmaceutical formulation according to the present disclosure has a pH about 4.5 to 7.0 and contains less than 0.74 endotoxin units per ml and is wherein in said deuterated trehalose at least one hydrogen atom attached to a carbon atom is replaced by a deuterium atom.

In some embodiments the aqueous pharmaceutical formulation as herein defined is adapted for enteral administration. In certain embodiments the aqueous pharmaceutical formulation as herein defined is adapted for oral or intragastric administration.

In other embodiments the aqueous pharmaceutical formulation in accordance with the present disclosure is wherein the concentration of the deuterated trehalose is between about 0.1% (w/v) to about 50% (w/v). In specific embodiments the concentration of the deuterated trehalose is about 10% (w/v).

In further embodiments the aqueous pharmaceutical formulation as herein defined has an osmolality of about 280-330 mOsm/Kg.

In yet further embodiments the aqueous pharmaceutical formulation as herein defined is administered at a frequency between once daily to once per month.

In still further embodiments the aqueous pharmaceutical formulation as herein defined is wherein the therapeutically effective amount of deuterated trehalose or a mixture of several deuterated trehaloses, optionally together with non-deuterated trehalose is from about 1 gram to about 100 gram for each daily injection and no more than about 1 gram/kg body weight of said subject per day.

In some embodiments the aqueous pharmaceutical formulation according to the present disclosure is administered once daily at from about 1 mg/kg/day to about 1 gram/kg/day of deuterated trehalose or a mixture of several deuterated trehaloses, optionally together with non-deuterated trehalose.

In other embodiments the aqueous pharmaceutical formulation as herein defined is administered at a frequency of between once daily to once per month at a dose of about 5 to about 35 grams of deuterated trehalose or a mixture of several deuterated trehaloses, optionally together with non-deuterated trehalose comprised therein.

In yet other embodiments the aqueous pharmaceutical formulation according to the present disclosure is for administration once daily, once every other day, twice a week, once a week, once every two weeks, once every three weeks or once a month.

In further embodiments the aqueous pharmaceutical formulation as herein defined is wherein the dose is 5, 8, 15, 30, 40 or 50 grams.

In still further embodiments the aqueous pharmaceutical formulation as herein defined is wherein the rate of administration is such that the maximum endotoxin level is less than 5 endotoxin units per kilogram of body weight per hour.

In some embodiments the aqueous pharmaceutical formulation of the present disclosure is adapted for intravenous administration and wherein said administration is completed within from about 75 to about 120 minutes, specifically within less than 90 minutes.

In another one of its aspects the present disclosure provides a solid dosage form for oral administration, comprising deuterated trehalose or a mixture of several deuterated trehaloses, optionally together with non-deuterated trehalose as active ingredient, and further optionally comprising at least one of carrier, additive or excipient.

In yet another one of its aspects the present disclosure provides the aqueous pharmaceutical formulation or the solid dosage form as herein defined for use in a method of treating a disease associated with abnormal protein aggregation and/or inclusion bodies formation in myocytes, neurons and other cells or extracellular compartments or for alleviating a sign or symptom associated therewith in a human subject in need thereof.

The present disclosure further provides a kit comprising:
(a) pharmaceutically acceptable deuterated trehalose or active derivative thereof or a mixture of several deuterated trehaloses, and optional non-deuterated trehalose;
(b) at least one pharmaceutically acceptable additive, carrier, excipient and diluent;
(c) means for preparing an injectable aqueous solution of the deuterated trehalose by mixing said deuterated trehalose with at least one of said additive, carrier, excipient and diluent;
(d) means for parenterally administering said injectable solution to a patient in need;
(e) instructions for use.

### DETAILED DESCRIPTION OF EMBODIMENTS

The presently disclosed subject matter is based on the approach that deuterated trehalose, in particular a deuterated trehalose in which the α-α link is deuterated in any manner, i.e. at any one of the adjacent C-bonded hydrogen atoms, which exhibits a decreased susceptibility to hydrolysis by trehalase in vitro, can be particularly suitable for administration to a subject in need, specifically oral and parenteral administration, and can allow for better absorption into the blood and target organ/tissues, and longer residence time is these targets as compared to non-deuterated trehalose.

Increased stability of deuterated trehalose to hydrolyzation by trehalase, so far exhibited in vitro, should enable the use of smaller doses, and may result in higher plasma levels of the deuterated trehalose, and higher levels in target organs/tissues, such as nerve tissue in HD patients or muscle tissue in OPMD patients, and may also affect the bioavailability of the drug. Considering the fast GI degradation of trehalose by the gastric/intestinal enzyme, the use of deuterated trehalose for oral administration can be of particular interest, but in every route of administration it is expected to be advantageous, if but for the possibility to lower doses and/or frequency of administration.

Trehalose (also known as mycose or tremalose) is a natural alpha-linked disaccharide formed by an α,α-1,1-glucoside bond between two α-glucose units. Trehalose is known for its ability to sustain and preserve a wide array of biological molecules. As detailed above, trehalose has been used in a variety of research applications and is contained as an inert additive/excipient in several commercially available therapeutic products, including Herceptin, Avastin, Lucentis, and Advate, where it serves mainly as a protein stabilizer. Although trehalose is widely used as an excipient/additive together with another active ingredient, its use as a therapeutically active ingredient per se is rather exceptional, all the more so for deuterated trehalose.

It was reported that trehalose is capable of inhibiting intra-cellular aggregation of abnormal proteins associated with neurodegenerative diseases and myopathies due to its protein stabilization and autophagy enhancement capabilities. The leading example is the aggregation-suppressing effect of trehalose on the mutant huntingtin, a polyglutamine protein causing Huntington's disease (HD) in a mouse model of HD [7]. This publication demonstrates that oral administration of trehalose to transgenic HD mice (0.2%-5% trehalose in drinking water consumed spontaneously in the course of 5-9 weeks) led to inhibition of formation of intra-nuclear huntingtin aggregates in the brain and liver and, more importantly led to improvement of HD-related motor symptoms.

However, therapeutic use of trehalose, especially in the context of neuronal and muscular diseases, is significantly precluded by the fact that trehalose is hydrolyzed by the intrinsic enzyme trehalase at the epithelial brush border in the small intestine [12, 13], due to which only a small fraction (0.5%) of any enterally administered dose reaches blood stream, neuronal or muscle tissues, as mentioned above, where it is further metabolized by blood, renal and liver trehalases.

Trehalase (α,α-trehalase, α,α-trehalose glucohydrolase, EC 3.2.1.28) is an anomer-inverting (α→β) glucosidase, catalyzing the hydrolysis of the α-glucosidic O-linkage of α,α-trehalose to liberate equimolecular quantities of β-glucose and α-glucose [14].

The present disclosure aims at introducing a novel therapeutic regime using administration to a subject in need of deuterated trehalose, which is more resistant to hydrolysis by the enzyme trehalase, thereby achieving higher bioavailability and therapeutic efficacy in the treatment of myopathic and neurodegenerative diseases associated with abnormal protein aggregation, specifically polyalanine, polyglutamine and tauopathies, disorders, and HD and OPMD in particular.

Thus, the presently disclosed subject matter provides a method for treating or alleviating a disease associated with abnormal protein aggregation and/or inclusion bodies formation in myocytes, neurons and other cells or extracellular compartments, or at least one symptom associated therewith, in a human subject in need thereof comprising administering to said subject a therapeutically effective amount of deuterated trehalose or a pharmaceutical formulation comprising a therapeutically effective amount of deuterated trehalose, wherein in said deuterated trehalose at least one hydrogen atom attached to a carbon atom is replaced by a deuterium atom.

Isotopes are atoms which have nearly identical properties but which have different masses due to changes in the number of neutrons in their nuclei [14]. Deuterium is an isotope of hydrogen with a nucleus comprising one neutron and one proton. Kinetic isotope effects are the observed changes in the rate of reaction that occur when deuterium is substituted for hydrogen or vice versa. Deuterium isotope effects result from the greater energy required to break a covalent bond to deuterium versus a covalent bond to hydrogen. One of the challenges of incorporating deuterium into a pharmaceutical composition is the possibility of deuterium/hydrogen exchange within the physiological environment, eviscerating the effect of the compound.

The present disclosure encompasses trehalose molecules in which at least one hydrogen atom attached to a carbon atom is replaced by a deuterium atom. As indicated above, trehalase catalyzes the hydrolysis of the α-glucosidic O-linkage of α,α-trehalose to liberate equimolecular quantities of β-glucose and α-glucose. Therefore, a substitution of a hydrogen atom by a deuterium atom on the carbon atom bound to the oxygen atom that forms the O-linkage will reduce the hydrolysis rate of the deuterated trehalose by trehalase.

Some non-limiting examples of trehalose molecules in which a hydrogen atom was replaced by a deuterium atom on the carbon atom bound to the oxygen atom that forms the O-linkage trehalose in each one of the glucose monomers of the trehalose molecules are:
α,α-[1,1′-²H₂]trehalose (also named (α-D[1-²H]Glcp-(1⇄1)-α-D-[1-²H]Glcp)) having a structure according to Formula I:

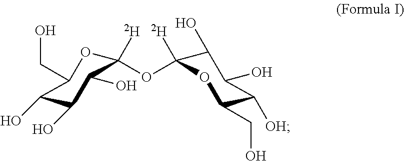

α,β-[1,1′-²H₂]trehalose (also named (α-D[1-²H]Glcp-(1⇄1)-β-D-[1-²H]Glcp)) having a structure according to Formula II:

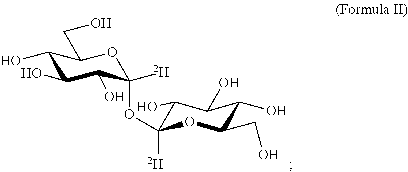

β,β-[1,1′-²H₂]trehalose (also named (β-D-[1-²H]Glcp-(1⇄1)-β-D-[1-²H]Glcp)) having a structure according to formula III:

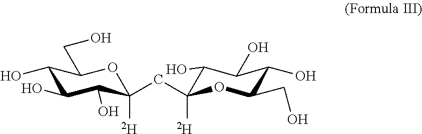

α,α-[UL-²H₁₄]trehalose (also named (α-D[UL-²H₇]Glcp-(1⇄1)-α-D-[UL-²H₇]Glcp)) having a structure according to formula IV

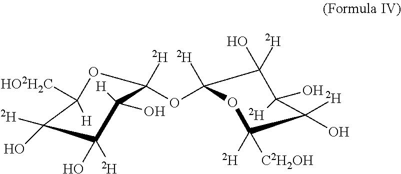

In specific embodiments the deuterated trehalose molecule encompassed by the present disclosure is α,α-deuterated trehalose, more specifically α,α-[1,1′-²H₂]trehalose having a structure according to Formula I:

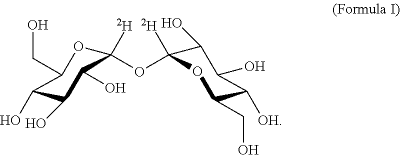

In other specific embodiments according to the present disclosure, including but not limited to methods of treatment and various uses, the deuterated trehalose is selected from:
α,α-[1,1′-²H₂]trehalose having a structure according to Formula I:

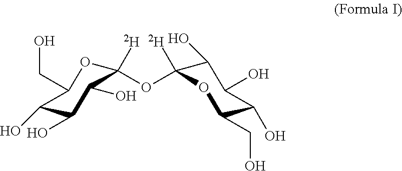

α,β-[1,1′-²H₂]trehalose having a structure according to Formula II

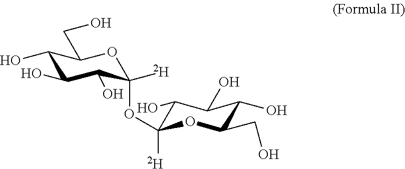

β,β-[1,1′-²H₂]trehalose having a structure according to formula III

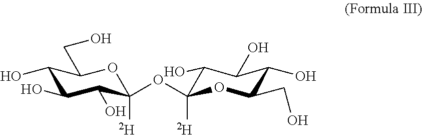

α,α-[UL-²H₁₄]trehalose having a structure according to formula IV

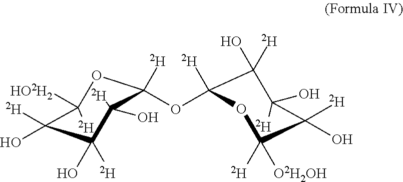

α,β-[UL-²H₁₄]trehalose, having a structure according to formula V

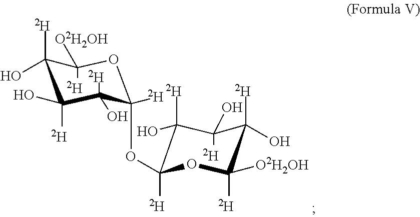

and
β,β-[UL-²H₁₄]trehalose having a structure according to formula VI

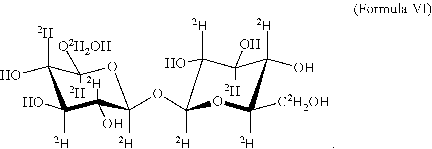

In other embodiments the deuterated trehalose molecule encompassed by the present disclosure is a molecule in which one hydrogen atom was replaced by a deuterium atom on the carbon atom bound to the oxygen atom that forms the O-linkage trehalose in one of the glucose monomers of the trehalose molecules, for example:

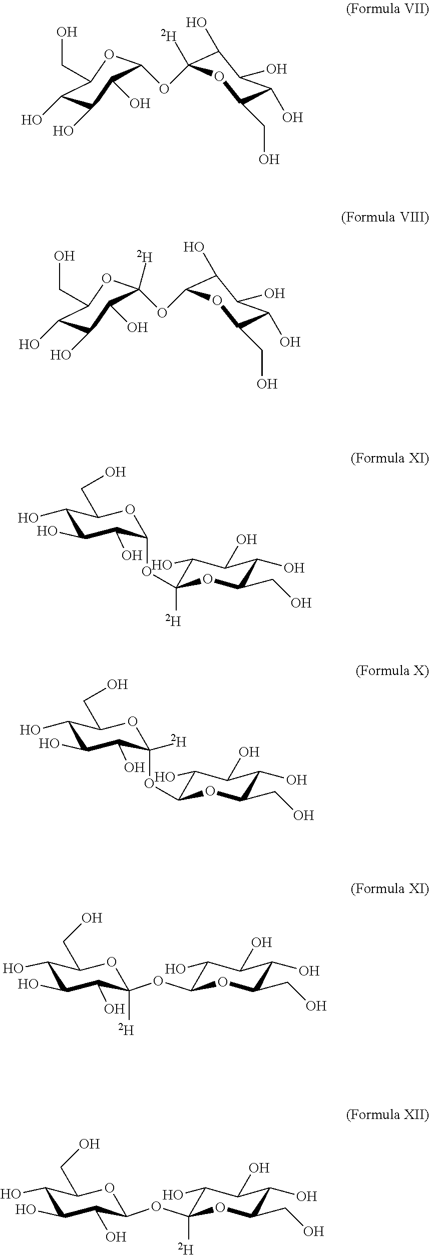

As indicated above, the presently disclosed subject matter provides a method for treating or alleviating a disease associated with abnormal protein aggregation and/or inclusion bodies formation in myocytes, neurons and other cells or extracellular compartments, or at least one symptom associated therewith. As herein defined the term “disease associated with abnormal protein aggregation and/or inclusion bodies formation in myocytes, neurons and other cells or extracellular compartments” refers to any disease associated with protein aggregation or protein misfolding, where the common underlying biological feature of these diseases being the aggregation of certain peptides and/or proteins, thereby generating a cascade of pathological events, including the secondary aggregation of various other proteins and the consequent failure of protein homeostasis to preserve normal biological function.

The term “treating or alleviating” as herein defined refers to achieving a therapeutic effect, ameliorating, relieving or reducing the severity and/or frequency of at least one sign or symptom associated with diseases as herein defined, elimination of signs or symptoms and/or underlying cause, prevention of the occurrence of symptoms and/or their underlying cause (e.g., prophylactic therapy), improvement or remediation of damage and eliminating or reducing the extent of protein aggregation.

Symptoms associated with the diseases as herein defined include, but are not limited to, drooping eyelids (a condition known as “ptosis”), difficulty in swallowing (called “dysphagia”), muscle fatigue, movement/motion disorders and cognitive disorders to name but few.

In particular, where the disease to be treated is Huntington's disease (HD) the formulations and methods described herein are useful in the treatment of the signs and symptoms of HD. Signs and symptoms of HD include, but are not limited to chorea, psycho-neurological deterioration, gait abnormalities, ataxia and cognitive disorders.

Where the disease to be treated is oculopharyngeal muscular dystrophy (OPMD), the formulations and methods described herein are useful in the treatment of the signs and symptoms of OPMD, including, but not limited to severe dysphagia, ptosis, tongue atrophy and weakness, lower and upper limb proximal weakness, dysphonia, limitation in upward gaze and facial muscle weakness.

As known in the art, ample diseases and disorders are defined as “disease associated with abnormal protein aggregation and/or inclusion bodies formation in myocytes, neurons and other cells or extracellular compartments”. For example, the presently disclosed subject matter encompasses treating or alleviating a disease associated with abnormal protein aggregation which is a neurodegenerative disorder, a poly-alanine or a poly-glutamine aggregation disorder, a protein codon reiteration disorder, a myopathy, and a tauopathy, to name but few.

The term “neurodegenerative disorder” as herein defined refers to hereditary or sporadic conditions characterized by progressive nervous system dysfunction, specifically disorders of this group that are associated with formation of abnormal protein aggregates, also known as inclusion bodies formation. Examples for a neurodegenerative disorder which is associated with abnormal protein aggregation are Amyotrophic lateral sclerosis (ALS), Alzheimer's disease, Parkinson's disease and others.

The terms “poly glutamine”, “poly-alanine aggregation disorder” or “protein codon reiteration disorder” as herein defined refer to disorders associated with formation of intracellular polyglutamine or polyalanine aggregates, preferably referring to Huntington's disease (HD), oculopharyngeal muscular dystrophy (OPMD), spinal and bulbar muscular atrophy (SBMA), dentatorubral-pallidoluysian atrophy (DRPLA) and spinocerebellar ataxia (SCA).

The term “myopathy” as herein defined refers to inherited or acquired degenerative disease involving atrophy of the muscle fibers, in the context of present disclosure particularly referring to OPMD.

The term “tauopathy” as herein defined refers to neurodegenerative diseases associated with tau-pathology, prototypic intracellular aggregation of tau microfilaments, in the context of present disclosure particularly referring to Pick's disease, corticobasal degeneration (CBD), progressive supranuclear palsy (PSP) and frontotemporal dementia and parkinsonism linked to chromosome 17 (FTDP-17).

Tauopathies are known as diseases caused by mutations leading to misfolding of Tau microtubule-associated protein that binds and stabilizes microtubules in neuronal cells. Tau pathology is a prominent feature of the sporadic Alzheimer's disease (AD), but is also seen in a variety of other related neurodegenerative diseases, such as Pick's disease, Corticobasal degeneration (CBD), Progressive supranuclear palsy (PSI)) and Frontotemporal dementia and parkinsonism linked to chromosome 17 (FTDP-17). More than 30 different inherited mutations or nucleotide substitutions in the FTDP-17 gene on chromosome 17q21 have been related to neurodegenerative disease manifesting a prototypic intracellular aggregation of tau microfilaments. Tau mutation and by analogy tau dysfunction in inherited and in sporadic diseases may be pathogenic through mechanisms involving both loss of function (decreased microtubules stabilization) and toxic gain of function (increased fibril formation).

Thus in the above and other embodiments the disease is any one of poly-glutamine aggregation disorder, poly-alanine aggregation disorder and a tauopathy.

In the above and other embodiments the disease is as herein defined is any one of Huntington's disease, oculopharengeal muscular dystrophy (OPMD), spinocerebellar ataxias (SCA), Friedreich's ataxia, spinal and bulbar muscular atrophy (SBMA), Parkinson's disease, Alzheimer's disease and amyotrophic lateral sclerosis (ALS), dentatorubral-pallidoluysian atrophy (DRPLA). Pick's disease, Corticobasal degeneration (CBD), Progressive supranuclear palsy (PSP) and Frontotemporal dementia and parkinsonism linked to chromosome 17 (FTDP-17).

In some embodiments the disease is as herein defined is Huntington's disease. As indicated above and as known in the art, Huntington's disease (HD) is a neurodegenerative genetic disorder that affects muscle coordination and leads to cognitive decline and psychiatric problems.

In other embodiments the disease is as herein defined is oculopharengeal muscular dystrophy (OPMD).

As indicated above, HD is associated with expansions of a homopolymeric stretch of polyglutamine (poly Q). It is a neurodegenerative genetic disorder that affects muscle coordination and leads to mental decline and behavioral symptoms. The disease is caused by an autosomal dominant mutation in either of an individual's two copies of the Huntingtin gene, encoding the protein “huntingtin”. Expansion of a CAG (cytosine-adenine-guanine, encoding glutamine) triplet repeat stretch within the Huntingtin gene results in a different form of the protein, which gradually damages cells in the brain. A child of an affected person typically has a 50% chance of inheriting the disease. Oral administration of trehalose, a disaccharide known for its protein stabilizing effect, has been shown in HD mouse model to reduce polyglutamine aggregates, improve motor dysfunction and extend survival [7]. More recently, these findings have been reproduced upon oral administration of trehalose in a mouse model of OPMD [8].

As indicated above, OPMD is caused by inherited abnormal expansions of polyalanine in the poly A binding protein nuclear 1 protein (PABPN1) leading to PABPN1 misfolding and formation of tubule-filamentous inclusions/aggregates in nuclei of the affected muscle cells. Oral administration of trehalose to transgenic OPMD mice (2% trehalose in drinking water consumed spontaneously during 4-6 months) was shown to significantly reduce aggregation formation and toxicity of the mutant PABPN1 in myocytes. Overall, oral trehalose was shown to delay the OPMD onset, attenuate the disease phenotype, decrease polyalanine protein aggregate formation and decrease cell death, thus suggesting that trehalose may be a potent anti-aggregation therapy for OPMD and other protein codon reiteration disorders.

It is suggested, that like trehalose, deuterated trehalose can also be delivered to muscle, which is the target organ in treatment of various diseases and disorders as herein defined, for example but not limited to OPMD. It is further suggested that deuterated trehalose can be successfully delivered to nerve cells, which are target organs in the treatment of various diseases and disorders as herein defined, for example but not limited to HD.

Huntington's disease and OPMD share a common genetic basis as both are caused by inherited trinucleotide expansion mutations in a disease causative gene. The HD gene (HTT), located on chromosome 4p16, contains repetitive trinucleotide sequences (CAG)n encoding for a polyglutamine (poly Q) stretch. While the normal HTT gene variants contain 7-35 repeats, HD-related variants are above that range and the extent of repeat expansions is correlated to the severity and earlier onset of HD symptoms (the effect termed anticipation). Analogous (CAG)n repeat expansions mutations were associated with other polyglutamine disorders, such as SBMA, DRPLA and several ataxias belonging to the group of SCA. Similarly, the OPMD-related gene, PABPN1 on chromosome 14q11 contains trinucleotide repeats (CGC)n encoding polyalanine (poly A), wherein (CGC)6 repeats is the normal threshold above which OPMD is anticipated with an increased number of repeats.

In view of the apparent lack of medical therapy for HD, OPMD and other related disorders apart from medications that lessen some motor and psychiatric symptoms, therapies that directly interfere with the disease-causing mechanisms are sorely needed.

In some embodiments the disease is as herein defined is spinocerebellar ataxia (SCA). As known in the art there are many types of spinocerebellar ataxia, with the most common Spinocerebellar ataxia including Friedreich's ataxia, SCA 1, 2, 3, 4, 6, 8, 17 and more. Many SCAs fall under the category of polyglutamine diseases, which are caused when a disease-associated protein (i.e., ataxin-1, ataxin-3, etc.) contains a glutamine repeat beyond a certain threshold.

In other embodiments the disease as herein defined is spinal and bulbar muscular atrophy (SBMA). The disease SBMA, also known as spinobulbar muscular atrophy, bulbo-spinal atrophy, X-linked bulbospinal neuropathy (XBSN), X-linked spinal muscular atrophy type 1 (SMAX1), and Kennedy's disease (KD), as known in the art, is a debilitating neurodegenerative disease resulting in muscle cramps and progressive weakness due to degeneration of motor neurons in the brain stem and spinal cord.

In further embodiments disease is as herein defined is Parkinson's disease, Alzheimer's disease or amyotrophic lateral sclerosis (ALS). Parkinson's disease is a degenerative disorder of the central nervous system; Alzheimer's disease (AD) is manifested by short term memory loss, confusion, irritability, aggression, mood swings, trouble with language, and long-term memory loss; and amyotrophic lateral sclerosis (ALS, also referred to as motor neuronal disease or Lou Gehrig's disease) is a neurodegenerative disease characterized by rapidly progressive weakness due to muscle atrophy and muscle spasticity, difficulty in speaking (dysarthria), swallowing (dysphagia), and breathing (dyspnea).

As indicated above, the presently disclosed subject matter provides a method for treating or alleviating a disease as herein defined in a human subject in need thereof comprising administering to said subject a therapeutically effective amount of deuterated trehalose or a pharmaceutical formulation comprising same.

In the above reports of trehalose as a therapeutic agent for diseases associated with protein aggregation, the studies were performed in mice, the only species that has a specialized pinocytotic mechanism for absorption of intact trehalose. Other species (rats, dogs, cats, human and others) cannot absorb orally administered trehalose even at very large amounts, due to the extensive breakdown of trehalose in intestine, blood, liver and kidneys. Applicant's co-pending WO 2014/181333 [11] is directed to a therapeutic regimen using trehalose that is administered to a subject in need by parenteral administration. Presently disclosed is the use of deuterated trehalose in the treatment of diseases associated with protein aggregation, such as Poly A, Poly Q and tauopathies, where lower doses and/or less frequent administrations are suggested.

In some embodiments the deuterated trehalose or a pharmaceutical formulation comprising thereof is administered parenterally.

In other embodiments the pharmaceutical formulation as herein defined is an injectable solution for parenteral administration.

In yet other embodiments the deuterated trehalose or a pharmaceutical formulation comprising thereof as herein defined is administered enterally, specifically by oral administration.

In still further embodiments the pharmaceutical formulation for enteral administration is an aqueous solution. In yet further embodiments the pharmaceutical formulation for enteral administration is a solid dosage form.

The term “subject in need thereof” refers to a subject suffering from a disease as herein defined, for example but not limited to HD or OPMD or another polyglutamine or polyalanine aggregation disorder.

The therapeutically effective amount of deuterated trehalose can be from about 1 mg to about 2,000 mg/Kg body weight of the subject per day. The deuterated trehalose can be comprised as active ingredient in a pharmaceutical formulation suitable for oral or for parenteral administration. Administration can be based on a daily regime as a single dose or multiple doses by a single parenteral administration or as multiple doses by multiple parenteral administrations, respectively. Alternatively or additionally, administration can be periodic, for example every other day, three times weekly, twice weekly, once weekly, or once monthly, and frequency of administration can be varied according to the patient's condition. Alternatively, administration can be based on a daily regime as a single dose or multiple doses by a single oral administration or as multiple doses by multiple oral administrations, respectively. Also where administration is oral, administration can be periodic, for example every other day, three times weekly, twice weekly, once weekly, or once monthly, and frequency of administration can be varied according to the patient's condition. Still alternatively, administration can be by combined mode, for example oral and parenteral.

The term “parenterally” as herein defined refers to a route of administration where the desired effect is systemic and the active agent (herein defined as deuterated trehalose), is administered by routes other than the digestive tract, for example intravenous, intramuscular and intraperitoneal administration.

The term “orally” as herein defined refers to a route of administration where the active agent (herein defined as deuterated trehalose), is administered enterally, namely through the digestive tract, for example, by oral or intragastric administration.

As mentioned above, trehalose is known in the art as a lyoprotectant, specifically of proteins such as, but not limited to antibodies. As such the safety and toxicity of trehalose has been extensively investigated, and the substance was found to be safe when administered both orally and intravenously, in doses that are substantially higher than the intended therapeutic dose. It is also known that deuterium has remarkably low systemic toxicity, and it is expected that humans can tolerate high levels of deuterium in body fluids. Deuterated trehalose is therefore to be a safe drug for human use.

As shown in the Examples below, pharmaceutical formulations comprising deuterated trehalose as sole active ingredient are parenterally and orally administered resulting in relatively high plasma, as well as nerve and muscle levels.

Thus, in some embodiments the pharmaceutical formulation as herein defined comprises deuterated trehalose as sole active ingredient, and optionally further comprises at least one pharmaceutically acceptable additive, carrier, excipient or diluent.

In other words, in the presently disclosed subject matter deuterated trehalose is the only active agent or ingredient. Notwithstanding the above, deuterated trehalose or a pharmaceutically formulation comprising same may be used in combination with other therapies or treatments for treating or alleviating a disease associated with abnormal protein aggregation.

Trehalose is a naturally occurring disaccharide comprised of a 1,1 linkage of two D-glucose molecules. It is a non-reducing sugar that is not easily hydrolyzed by acid. Its molecular formula is C₁₂H₂₂O₁₁ and its molecular weight is 342.31 Dalton. Other names used to describe the α, α form, the isomer commonly referred to as ‘trehalose’, are α,α-Trehalose, α-δ-glucopyranosyl, α-δ-glucopyranoside, mushroom sugar, mycose.

The term “trehalose” as herein defined refers to a disaccharide glucose α-G-glucopyranosyl α-D-glucopyranoside. The term trehalose also refers to any active derivative of trehalose, for example hydrides and salts thereof.

The term “deuterated trehalose” as herein defined refers to a disaccharide glucose α-G-glucopyranosyl α-D-glucopyranoside, in which at least one hydrogen atom bonded to a carbon atom is replaced by a deuterium atom. Specific examples of deuterated trehalose are trehalose molecules in which at least one hydrogen atom bonded to a carbon atom adjacent to the oxygen atom of the glucose-glucose glycosidic bond is replaced by a deuterium atom, and trehalose molecules in which each of the hydrogen atom bonded to the two carbon atoms adjacent to the oxygen atom of the glucose-glucose glycosidic bond is replaced by a deuterium atom. Non-limiting examples for deuterated trehalose molecules encompassed by the present disclosure are given in Formulae I-XII. The term “deuterated trehalose” also refers to any active derivative of deuterated trehalose, for example hydrides and salts thereof.

It is to be noted that the in all aspect and embodiments of the presently disclosed subject matter “deuterated trehalose” also encompasses a mixture of several different deuterated trehaloses. Furthermore, the active ingredient used in the compositions and methods of the present disclosure is to be taken to mean also mixtures of at least one deuterated trehalose with non-deuterated trehalose (trehalose), at any desired ratio, for example, but not limited to mixtures comprising at least 1% of at least one deuterated trehalose with up to 99% trehalose, for example at least 1%, 2%, 5%, 10%, 15%, 20%, 30%, 40%, 50%, 60%, 70%, 80, 90%, 99% w/w of at least one deuterated trehalose, with up to 99%, 98%, 95%, 90%, 85%, 80%, 70%, 60%, 50%, 40%, 30%, 20%, 10, and 1% non-deuterated trehalose.

As indicated above, trehalose is well known for its protein-stabilizing properties. It is used extensively in many applications as a stabilizer of frozen food, in freeze-drying of biological systems and cells, as a stabilizer of therapeutic parenteral proteins and as an excipient in tablets and IV solutions. Trehalose is recognized as a GRAS (Generally Regarded as Safe) food ingredient by the FDA and is listed on the USP-NF (United States Pharmacopoeia National Formulary), EP (European Pharmacopoeia) and JP (Japanese Pharmacopoeia).

Like all disaccharides, trehalose is metabolized at the epithelial brush border to two D-glucose molecules. Less than 0.5% of ingested trehalose is absorbed into the blood stream where it is further metabolized by plasma, liver and kidney by trehalase. Oral trehalose in amounts exceeding 40-50 gram per day causes diarrhea and bloating. Thus in order to achieve therapeutic amounts of trehalose in the muscle cells it was necessary to either circumvent the massive metabolism in the GI tract, for which the present applicant developed specific intravenous (IV) solutions of trehalose, or to increase its resistance to enzymatic or other degradation, as suggested herein, by replacing the hydrogen atom adjacent to the glycosidic bond of the trehalose by deuterium atoms. The deuterated trehalose is less susceptible to degradation, and therefore is suggested for enteral, specifically oral administration, as well as parenteral administration, apparently at reduced doses and/or frequency of administration.

The term “formulation” as herein defined refers to a composition comprising deuterated trehalose as active ingredient and optionally further comprising at least one additional active ingredient such as anti-inflammatory agent, and at least one pharmaceutically acceptable additive, carrier, excipient or diluents as well known in the art. This formulation may further comprise a trehalase inhibitor, i.e. competitive or other inhibitor of the trehalase enzyme. Such inhibitor is expected to also inhibit enzymatic degradation of deuterated trehalose.

Particular pharmaceutical formulations are suitable for parenteral administration, specifically injectable solutions, as well as for enteral, specifically oral administration. The concentration of deuterated trehalose in said formulation is between about 0.1% (weight/volume) to about 50% (weight/volume), more specifically wherein the concentration of deuterated trehalose in the formulation as herein defined is, but is not limited to, about 8%, 9%, 10%, 11%, 12%, 13%, 14%, 15%, 20% or 25% (weight/volume).

In specific embodiments the concentration of deuterated trehalose in the formulation as herein defined is about 10% (w/v).

Liquid formulations for oral administration may be in the form of solutions or syrups.

Other particular pharmaceutical formulations suitable for enteral, specifically oral administration, may be, for example solid dosage forms, such as tablets, capsules, caplets, lozenges, as known to the person of skill in the art. Solid forms such as for example tablets may be coated with an enteric coating.

When referring to trehalose and deuterated trehalose, the terms encompass not only the specified molecular entity but also its pharmaceutically acceptable, pharmacologically active analogs, including, but not limited to, salts, polymorphs, esters, amides, prodrugs, adducts, conjugates, active metabolites, and the like.

Thus the active agent, deuterated trehalose, may be administered in the form of the compound per se as well as in the form of a salt, polymorph, ester, amide, prodrug, derivative, or the like, provided the salt, polymorph, ester, amide, prodrug or derivative is suitable pharmacologically. Salts, esters, amides, prodrugs and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For any active agents that may exist in enantiomeric forms, the active agent may be incorporated into the present formulations either as the racemate or in enantiomerically pure form.

Salts are compounds that ionize in aqueous solutions and may be employed, for example, to adjust the tonicity of the solution. If the active agent is present in the form of a salt, additional salts may be added to the composition in order, for example, to effect ion exchange with the active agent. Salts suitable for use with the compositions described herein are known in the art and include, for example, lithium, sodium, potassium, calcium, and magnesium salts having appropriate counterions that may be selected from chloride, bromide, iodide, carbonate, phosphate, nitrate, silicate, sulfate, phosphite, nitrite, sulfite, and the like.

Buffers are compounds or solutions that are employed to aid in maintaining the concentration of an analyte within a desired range. For example, pharmaceutically acceptable pH buffers are used to maintain the acidity or basicity of a solution within a pharmaceutically acceptable range. Buffers for use in the compositions disclosed herein may be any known or hereafter discovered buffer.

Excipients are inactive ingredients that may be employed in the compositions described herein for a variety of reasons. A wide range of excipients are described in the literature (e.g., Rowe et al., Handbook of Pharmaceutical Excipients, McGraw Hill, 2006). Additives and diluents are well known in the art.

In some specific embodiments the pH of the formulation, particularly where designed for parenteral administration, is about 4.5 to 7.0, the osmolality of the formulation is about 280-330 mOsm/kg, the formulation contains less than 1.0, 0.9, 0.8, 0.7, 0.6, 0.5, 0.4, 0.3, 0.2, 0.1 or less endotoxin units per mL and the aqueous formulation is about 50%, 40%, 30%, 20%, 10%, 5% or less deuterated trehalose (w/v).

Thus the presently disclosed deuterated trehalose delivery systems, for example formulations comprising deuterated trehalose as sole active ingredient, can generally comprise a buffering agent (pH adjusting additives), an agent which adjusts the osmolality thereof, and optionally, one or more pharmaceutically acceptable carriers, excipients and/or additives as known in the art. Supplementary pharmaceutically acceptable active ingredients can also be incorporated into the formulations.

For formulations as herein defined administered as aqueous or other solvent-based dosage forms (e.g., for parenteral administration), a variety of liquid carriers may be used, in particular water or saline. Aqueous solutions may include salts, buffers, and the like. The carrier can be solvent or dispersion medium suitable for parenterally-administrable compositions containing, for example, water, saline, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. Water is an essential additive (or carrier).

Formulations and dosage forms designed for enteral, specifically for oral administration, can include flavoring agents.

Thus as used herein the term “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated. It is contemplated that the active agent can be delivered by any acceptable parenteral route and in any pharmaceutically acceptable dosage form.

For purposes of parenteral administration, formulations in suitable oil such as sesame or peanut oil or in aqueous propylene glycol can be employed, as well as sterile aqueous solutions of the corresponding water-soluble salts. Such aqueous formulations may be suitably buffered, if necessary, and the liquid diluent first rendered isotonic with sufficient saline or glucose. These aqueous formulations are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical formulations with a certain amount of active ingredient are known, or will be apparent in light of this disclosure, to those skilled in this art.

As described in the accompanying Examples, specific injectable, aqueous formulations comprising deuterated trehalose are prepared, to be used in studies of HD and OPMD patients. Solid dosage forms are also suggested.

Given the fact that over 99.5% of any administered trehalose is not absorbed into the blood stream, and that oral amounts of trehalose higher than 50 g a day in humans frequently cause diarrhea, bloating and discomfort as indicated above, it was shown in applicant's co-pending WO 2014/181333 [11] that the most effective way to ensure that adequate amounts of trehalose indeed reach the target cells/tissues is to circumvent the extensive gut metabolism and administer trehalose by an intravenous (IV) solution. Nonetheless, considering that the susceptibility of deuterated trehalose to enzymatic degradation is significantly lower than that of non-deuterated trehalose, it is currently suggested that oral formulations and dosage forms in which the active agent is deuterated trehalose can be administered also orally. Deuterated trehalose administered parenterally can be used at lower amounts and/or less frequent administrations.

Injectable solutions of deuterated trehalose for parenteral administration can be intravenously administered to HD or OPMD patients at a single administration (injection) during about 75 to 120 minutes, for example 90 minutes, every day, every other day, twice a week, once a week, once in 10 days, once every two weeks or once a month, for a defined number of administrations as will be determined by the attending physician.

Based on the pharmacokinetic profile of deuterated trehalose, initial once a week, 24-week IV therapy will enable the deuterated trehalose to enter the nerve cells, respectively muscle cells and potentially exert its therapeutic effect, previously demonstrated for non-deuterated trehalose in animal models. The amounts to be administered in the weekly injection are based on safety studies as well as the rate of metabolism and clearance from the blood so as to enable deuterated trehalose to reach nerve, respectively muscle cells before it is cleared from the blood.

The doses are calculated based on the expected plasma and tissue distribution of deuterated trehalose, effective concentration of deuterated trehalose in diseased cells and known safety doses in multiple animal studies.

Following initial treatment, once weekly administration can be chronic, at set time points, for spells of several or scores of weeks, for example additional consecutive 48 weeks, thus a total of 72 weeks of treatment, or administration can be periodic.

Deuterated trehalose is expected to effectively reach nerve and muscle cells/tissues by parenteral, specifically IV administration, as well as by enteral, specifically oral administration. Thus in some embodiments administration of deuterated trehalose or a pharmaceutical formulation or dosage form comprising the same may be in any one of intravenous, intramuscular and intraperitoneal administration, as well as oral and intra-gastric administration.

The purified deuterated trehalose designed for intravenous administration is substantially free of contaminants in the enzymatically prepared starting material trehalose, such as organic solvents used in the enzymatic process (e.g., ammonium, acetonitrile, acetamide or alcohols), TFA, ether or other contaminants. In this context “substantially” free of contaminants means that the contaminant content of any residual peptide originating from the enzymatic preparation process of the starting material trehalose at the end of the purification process is preferably less than 0.5%, less than 0.3%, less than 0.25%, less than 0.1%, less than 0.05%, less than 0.04%, less than 0.03%, less than 0.02%, less than 0.01%, less than 0.005%, less than 0.003%, or less than 0.001% of the total weight of the final deuterated trehalose. The content of contaminants can be determined by conventional methods such as gas chromatography.

In specific embodiments, the residual solvents in the purified deuterated trehalose designed for intravenous administration are less than the limits set in the ICH guidelines, e.g., Impurities: Guideline for Residual Solvents Q3C(R5) (available at the world wide web (www) tga.gov.au/pdf/euguide/ich822602006.pdf) [16]). For example, the purified trehalose contains <5000 ppm ethanol (e.g., <140 ppm), and/or <3000 ppm methanol, and deuterated trehalose is to contain no more than these amounts that are accepted for purified non-deuterated trehalose.

Endotoxins (also known as lipopolysaccharides (LPS) and lipoglycans) are large molecules consisting of a lipid and a polysaccharide composed of 0-antigen, outer core and inner core joined by a covalent bond; they are found in the outer membrane of Gram-negative bacteria, and elicit strong immune responses in animals. Endotoxins are well-known contaminants in substances purified by using bacterial systems and their removal is thus crucial for safety of using therapeutic formulations comprising such substances.

Presently disclosed formulations of deuterated trehalose designed for intravenous administration should comprise less than 5 endotoxin units per kilogram of body weight of a patient administered with the formulation, per hour of administration. Thus in some embodiments the pharmaceutical formulation as herein defined comprises less than 0.74 endotoxin units per ml solution.

Therapeutically effective amount of deuterated trehalose as herein defined will depend on a number of factors and will vary from subject to subject and may be determined by considerations well known to a skilled person in the field of the invention (e.g. a skilled physician). Such factors include the severity of the symptoms, the patient's age, weight and general condition, and the judgment of the prescribing physician.

Generally, by the term “therapeutically effective amount” it is meant a nontoxic but sufficient amount of deuterated trehalose to provide the desired effect, namely the treating or alleviating a disease associated with abnormal protein aggregation and/or inclusion bodies formation in neurons, myocytes and other cells or extracellular compartments.

The term “therapeutically effective amount or pharmaceutical composition comprising thereof” is to be understood as the amount of deuterated trehalose comprised in the administered formulation. The amounts of any liquid formulations itself may vary according to the concentration of the deuterated trehalose comprised therein (namely, such that the concentration of deuterated trehalose in liquid formulations is between about 0.1% (w/v) to about 50% (w/v) and such that the maximum endotoxin level in the administered formulation is less than 5 endotoxin units per kilogram of body weight per hour).

Therapeutically effective amount of deuterated trehalose formulation in parenteral administration refers to an amount of from about 1 gram to about 100 gram for each daily injection and no more than 1 g/kg body weight of said subject per day of a treated human subject. Amount may considerably vary. Thus the range can be from each of 10, 20, 50, 75, 100, 150, 200, 300 mg/Kg body weight per day up to each of 400, 450, 500, 550, 600, 650, 700, 750, 800, 850, 900, 950 and 1000 mg/Kg body weight per day

In other embodiments, therapeutically effective amount of parenterally administered deuterated trehalose as herein defined is from about 1 gram to about 100 gram for each single injection and no more than about 1 gram/kg body weight of said subject per day.

For formulation administered enterally, specifically orally, the amount of deuterated trehalose is from about 1 mg/kg to about 2 g/Kg body weight of the patient per day, for example from each of 1, 5, 10, 20, 50, 75, 100, 150, 200, 300 mg/Kg body weight per day up to each of 400, 450, 500, 550, 600, 650, 700, 750, 800, 850, 900, 950, 1000, 1200, 1500 and 2000 mg/Kg body weight per day.

The terms “injection” and “infusion” are used interchangeably and refer to parenteral administration of the formulation as herein defined.

The therapeutically effective amount of deuterated trehalose or pharmaceutical composition or formulation comprising thereof is administered parenterally, and can be administered as a single dose or multiple doses, which may be identical or different, by a single administration per day, or multiple doses can be parenterally administered by multiple daily or weekly or monthly or more prolonged administrations.

A therapeutically effective amount of deuterated trehalose or pharmaceutical composition according to the above, administered IV, IP or IM, as a single dose or multiple doses, which may be identical or different, by a single administration per day, or optionally as multiple doses, which may be identical or different, by multiple administrations per day, or further optionally in a dosing regimen of equal doses, or gradually increasing doses, or gradually decreasing doses, or further chronically or periodically.

In other embodiments, the therapeutically effective amount of deuterated trehalose or pharmaceutical composition or formulation comprising thereof is administered enterally, specifically orally, and can be administered as a single dose or multiple doses, which may be identical or different, by a single administration per day, or multiple doses can be enterally administered by multiple daily or weekly or monthly or more prolonged administrations.

A therapeutically effective amount of deuterated trehalose or pharmaceutical composition according to the above, administered enterally, as a single dose or multiple doses, which may be identical or different, by a single administration per day, or optionally as multiple doses, which may be identical or different, by multiple administrations per day, or further optionally in a dosing regimen of equal doses, or gradually increasing doses, or gradually decreasing doses, or further chronically or periodically.

Optimal dosing schedules may be calculated from measurements of drug accumulation in the body of the patient. Optimal dose can be determined by dosing methodologies and repetition rates. In general, dosage is calculated according to body weight, and may be given once or more daily, weekly, monthly or yearly, or even once every few years. Persons of ordinary skill in the art can readily estimate repetition rates for dosing based on measured residence times and concentrations of the combined composition of the invention in bodily fluids or tissues. Following successful treatment, it may be desirable to have the patient undergo maintenance therapy to prevent the recurrence of the disease state, wherein the combined composition of the invention is administered in maintenance doses, once or more daily.

In some embodiments the therapeutically effective amount of deuterated trehalose or pharmaceutical formulation comprising same as herein defined is administered chronically.

The term “chronically” as herein defined refers to a constant regime of administration occurring at a predetermined frequency. Thus administration may be based for example on a daily, weekly or monthly regime as a single parenteral (specifically intravenous) or enteral (specifically oral) dose or multiple parenteral or enteral doses.

In some embodiments the frequency as herein defined is of between once daily to once per month, namely said therapeutically effective amount of deuterated trehalose or pharmaceutical formulation comprising thereof is administered at a frequency of between once daily to once per month.

In other embodiments the frequency as herein defined is every day (daily), namely seven times per week or six, five, four or three times per week, twice or once per week, once in every two weeks, once in every three weeks or once per month.

In specific embodiment the frequency as herein defined is once per week. In other embodiments the therapeutically effective amount of deuterated trehalose or deuterated trehalose comprised in said pharmaceutical formulation or dosage form is administered at a single administration once a week.

In the above and other embodiments the therapeutically effective amount of deuterated trehalose or deuterated trehalose comprised in said pharmaceutical formulation or dosage form is administered once daily at from about 1 mg/Kg/day to about 1 gram/Kg/day of deuterated trehalose, for example from about 10, 20, 30, 50, 75, 100, 250, 500 to about 1 gram/Kg/day of deuterated trehalose.

As indicated in the accompanying Examples, in a clinical study an aqueous injectable pharmaceutical formulation comprising deuterated trehalose is administered intravenously at a weekly dosing of 8, 15 or 30 gram per subject.

Thus in the above and other embodiments the therapeutically effective amount of deuterated trehalose or pharmaceutical formulation comprising the same is administered at a single dose of from about 5 to about 35 grams of deuterated trehalose, which may be administered once daily, once every other day, twice a week, once a week, once every two weeks, once every three weeks or once a month.

In further embodiments the therapeutically effective amount of deuterated trehalose or deuterated trehalose comprised in said pharmaceutical formulation is administered at a single dose of 5, 8, 15, 30, 40 or 50 grams of deuterated trehalose.

In other embodiments the therapeutically effective amount of deuterated trehalose or a pharmaceutical formulation comprising the same, specifically for parenteral administration is adapted for an injectable solution and wherein the rate of administration is such that the maximum endotoxin level is less than 5 endotoxin units per kilogram of body weight per hour.

By the term “rate of administration” it is meant the rate of infusion (or dosing rate).

In further embodiments the therapeutically effective amount of deuterated trehalose or a pharmaceutical formulation comprising the same, for parenteral administration, is adapted for intravenous administration and said administration is completed within less than about 75 to 120 minutes, for example but not limited to within 90 minutes. The period of administration will also depend on the volume/amount of the injectable deuterated trehalose formulation to be administered.

Thus in some embodiments administration of the therapeutically effective amount of deuterated trehalose comprised in said pharmaceutical formulation adapted for intravenous administration is completed within from about 75 to about 120 minutes, specifically within less than 90 minutes.

In still further embodiments the administration of deuterated trehalose or a pharmaceutical formulation comprising the same comprises a dosing regimen of equal doses, or gradually increasing doses, or gradually decreasing doses of deuterated trehalose or pharmaceutical formulation comprising the same.

In some embodiments the therapeutically effective amount of deuterated trehalose or pharmaceutical formulation comprising thereof is administered periodically.

By the term “periodically” it is meant that the administration of deuterated trehalose may be conducted at consecutive chronic administration regimens as herein defined which may be separated in time by periods of “no treatment” (or “drug holiday”, i.e. when the patient as herein defined stops taking the active agent for a certain period of time) according to the patient's condition.

Determining the efficacy of the treatment as herein defined may be performed by any method known to a person skilled in the art. For example, when the disease to be treated is HD, efficacy can be determined in association with any method known for diagnosing or treating HD. Alleviation of one or more signs or symptoms of HD indicates that the deuterated trehalose confers a clinical benefit. When the disease to be treated is OPMD, efficacy of the treatment may be performed in association with any known method for diagnosing or treating OPMD. Alleviation of one or more signs or symptoms of OPMD indicates that the deuterated trehalose confers a clinical benefit.

Thus in some embodiments the disease according to the presently disclosed subject matter is HD and in such case efficacy of treatment as herein defined may be performed by monitoring the patient's motor, cognitive, behavioral and functional assessments using for example the United Huntington Disease assessment scale (UHDRS).

The UHDRS is a standardized rating system used to quantify the severity of HD. Used clinically and in research, it measures the patient's abilities in four general areas: motor, cognitive, behavioral, and functional. The different portions of the test may be performed separately.

The following table summarizes the individual categories tested in the motor section of the UHDRS:

| Skill Category | Description |
|---|---|
| Ocular Pursuit | The ability of the patient to follow a finger with the eyes in both the horizontal and |
| | vertical directions |
| Saccade Initiation | The ability of the patient to turn the head in both the horizontal and vertical directions |
| Saccade Velocity | The speed at which the patient is able to turn the head both horizontally and vertically |
| Dysarthria | The presence of speech that is slurred, slow, and difficult to understand |
| Tongue Protrusion | The ability to stick out the tongue and the speed to which the task is completed |
| Finger Taps | The ability to tap the fingers of both hands (15 repetitions in 5 seconds is considered |
| | normal) |
| Pronation/Supination | The ability to rotate the forearm and hand such that the palm is down (pronation) and to |
| | rotate the forearm and hand such that the palm is up (supination) on both sides of the body |
| First-Hand-Palm | The ability to complete the sequence (making a fist, opening the hand palm down, and |
| Sequence | then rotating the hand palm up) more than 4 times in 10 seconds without cues is |
| | considered normal |
| Rigidity in arms | The severity to which the range of motion of the arms is limited |
| Bradykinesia | Slowness in initiation and continuation of movements |
| Maximal Dystonia | Abnormal muscle tone (measured separately in the extremities, face, and trunk) |
| Maximal Chorea | Involuntary jerky movements of the body (measured separately in the extremities, face, |
| | and trunk) |
| Gait | Walking with normal posture |
| Tandem Walking | The ability to walk in a straight line from heel to toe. The ability to do so regularly for 10 |
| | steps is considered normal |
| Retropulsion | The ability to stand after being pushed back |

In each category, patients are scored from 0 to 4, with 0 representing normal function, and 4 being the most severe dysfunction. The total score is the sum of the scores in the individual sub-categories. A higher UHDRS score indicates a more severe disease progression.

In other embodiments the disease according to the presently disclosed subject matter is OPMD and in such case efficacy of treatment as herein defined may be performed by monitoring the patient's weight; performing a “drinking test”, in which the patient is requested to drink 80 ml of ice-cold water, and the time which this volume has been fully consumed (in seconds) will be recorded; and by fiberoptic endoscopic evaluation of swallowing (FEES), which is a useful supplementary tool for studying dysphagia. The FEES procedure involves introducing a flexible fiberoptic endoscope transnasally to the patient's hypopharynx where the clinician can clearly view laryngeal and pharyngeal structures. The patient is then led through various tasks (e.g. food and liquid boluses) to evaluate the sensory and motor status of the pharyngeal and laryngeal mechanism. Information obtained from this examination includes ability to protect the airway, ability to sustain airway protection for a period of several seconds, ability to initiate a prompt swallow without spillage of material into the hypopharynx, timing and direction of movement of the bolus through the hypopharynx, ability to clear the bolus during the swallow, presence of pooling and residue of material in the hypopharynx, timing of bolus flow and airway protection, sensitivity of the pharyngeal/laryngeal structures and the effect of anatomy on the swallow.

Additional tests for evaluating efficaciousness of treatment of OPMD include the SWAL-QOL test (a 44 item tool that asks patients to rate several factors about 10 quality-of-life concepts related to swallowing on a 5 point scale) that was developed for measuring objectively a patient's perspective of swallowing and the muscle strength assessment (assessment of weakness of the proximal muscles, including “stair climb” test, “30 second sit-to-stand” test, “30 second weight arm raise” test, etc.).

The term “subject in need thereof” as herein defined is a subject suffering from a disease or disorder as herein defined, namely a disease associated with abnormal protein aggregation and/or inclusion bodies formation in myocytes, neurons and other cells or extracellular compartments.

In specific embodiments the subject in need in accordance with the present disclosure is diagnosed as suffering from HD or OPMD.

The present disclosure further provides deuterated trehalose or a pharmaceutical formulation comprising same, for use in a method for treating or alleviating a disease associated with abnormal protein aggregation and/or inclusion bodies formation in myocytes, neurons and other cells or extracellular compartments or at least one symptom associated therewith, in a human subject in need thereof, said method comprising administering to said subject a therapeutically effective amount of deuterated trehalose or a pharmaceutical formulation comprising the same, wherein in said deuterated trehalose at least one hydrogen atom attached to a carbon atom is replaced by a deuterium atom. In all aspects and embodiments, the deuterated trehalose as herein defined, per se or as the active ingredient in a composition or a formulation, is for use in a method for treating or alleviating a disease as herein defined.

The present disclosure further provides an aqueous pharmaceutical formulation comprising a therapeutically effective amount of deuterated trehalose as a sole active ingredient, wherein the formulation has a pH about 4.5 to 7.0 and contains less than 0.74 endotoxin units per ml and wherein in said deuterated trehalose at least one hydrogen atom attached to a carbon atom is replaced by a deuterium atom.

In certain embodiments, the present disclosure provides a formulation comprising deuterated trehalose or a physiologically acceptable derivative thereof. In certain embodiments deuterated trehalose (or derivative thereof) or at least a portion of the deuterated trehalose or derivative thereof is formulated for sustained and/or controlled release and a portion of the deuterated trehalose or derivative thereof is formulated for immediate release when administered to a subject.

In certain embodiments, effective serum levels of the active ingredient deuterated trehalose or derivative thereof are achieved within from about 10 to about 20 or 30 or 40 or 50 or 60 minutes following deuterated trehalose administration. In certain embodiments, effective serum levels of the active ingredient deuterated trehalose are achieved within from about 5 to about 20 or 30 or 40 or 50 or 60 minutes following deuterated trehalose administration. In certain embodiments, effective serum levels of the active ingredient are achieved within from about 20 to about 20 or 30 or 40 or 50 or 60 minutes following deuterated trehalose administration. In certain embodiments, effective serum levels of the active ingredient are achieved within about 5, 10, 15, 20, 30, 40, 50 or 60 minutes following deuterated trehalose administration.

Applicant's co-pending WO 2014/181333 [11] presented innovative approaches for the administration of trehalose based on parenteral routes. These approaches provide for a rational design of delivery systems (e.g. formulations) with desired properties based on the meticulous selection of the carrier, e.g. appropriate surfactants/co-surfactants composition or micro/nano particles (such as liposomes or nano-liposomes) or polymer entrapping the active ingredients, or other additives or excipients, for the delivery system of interest.

The parenteral ways of administration of deuterated trehalose in accordance with the presently disclosed subject matter include subcutaneous and transdermal (diffusion through the intact skin) administration. In certain embodiments, the present formulations are administered by invasive modes of treatment such as by intravenous, intramuscular and like administration.

Another way of administration in accordance with the presently disclosed subject matter can make use of red blood cells loaded with high quantities of deuterated trehalose. It is currently known that loading red blood cells with high amounts of trehalose enhances their viability. Such red blood cells, highly loaded with deuterated trehalose can serve as a vehicle for intravenous delivery of deuterated trehalose to a subject in need, in accordance with the present disclosure.

Administration of deuterated trehalose for medical uses requires safe and efficient delivery systems. Presently disclosed formulations for parenteral as well as enteral administration are designed for safe delivery of deuterated trehalose. The delivery systems enhance efficiency and quality of deuterated trehalose absorption, and enable lower concentrations or amounts of deuterated trehalose to be delivered to a subject in a biologically active form. Present delivery systems are to provide for the direct access of the active substance deuterated trehalose to the tissues and thus provide immediate or near-immediate effects of deuterated trehalose to the subject in need thereof.

Accordingly, in certain embodiments, the presently disclosed subject matter provides pharmaceutical delivery systems for the improved administration of deuterated trehalose or physiologically active derivative thereof, comprising as the active ingredient said deuterated trehalose or physiologically active derivative thereof in a suitable carrier for fast restoration of relief of symptoms of the disease of the treated subject.

In certain embodiments, the drug delivery systems may provide the active substance in a controlled release mode. In certain embodiments, the drug delivery systems of the invention may further comprises at least one additional pharmaceutically active agent.

Presently disclosed liquid delivery systems can generally comprise a buffering agent, an agent which adjusts osmolality, and optionally, one or more pharmaceutically acceptable carriers, excipients and/or additives as known in the art. Supplementary pharmaceutically acceptable active ingredients can also be incorporated into the compositions. The carrier can be solvent or dispersion medium suitable for parenterally-administrable compositions containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

In certain embodiments, deuterated trehalose or a formulation comprising thereof is administered orally. Oral delivery systems of deuterated trehalose can be liquid, and similar to the systems designed for parenteral administration, or solid, such as tablets, capsules, lozenges and the like.

As indicated above, present deuterated trehalose delivery system can be administered in controlled-, sustained- or delayed-release formulations. Any controlled or sustained release method known to those of ordinary skill in the art may be used with the formulations and methods of the presently disclosed subject matter such as those described in Langer 1990 [17]. Such method comprises administering a sustained-release composition or a coated implantable medical device so that a therapeutically effective dose of the composition of the invention is continuously delivered to a subject of such a method. Sustained release may also be achieved using a patch designed and formulated for the purpose. Controlled or sustained-release compositions include formulation in lipophilic depots (e.g., fatty acids, waxes, oils). Also comprehended by the invention are particulate compositions coated with polymers (e.g., poloxamers or poloxamines). Sustained release formulae or devices, or any topical formulations, may additionally contain compositions to stabilize the composition or permeate physiological barrier such as skin or mucous membrane. Exemplary additional components may include any physiologically acceptable detergent, or solvent such as, for example, dimethylsulfoxide (DMSO).

In certain embodiments, the deuterated trehalose in the present compositions can be formulated for sustained or controlled release over a period of at least 0.5, 1, 2, 3, 4, 5, 6, 7, 8, 9, 10, 11 or 12 hours. In certain embodiments, the deuterated trehalose in the present compositions can be formulated for sustained or controlled release over a period of about 0.5, 1, 2, 3, 4, 5, 6, 7, 8, 9, 10, 11 or 12 hours. In certain embodiments, the deuterated trehalose in the present compositions can be formulated for sustained or controlled release over a period of between about 0.5 or 1 or 2 or 3 or 4 hours and about 5, 6, 7, 8, 9, 10, 11 or 12 hours. In certain embodiments, the deuterated trehalose in the present compositions can be formulated for sustained or controlled release over a period of between about 5 or 6 or 7 or 8 hours and about 9, 10, 11 or 12 hours.

In certain embodiments, the deuterated trehalose in the present compositions can be in immediate, fast of burst release form.

In certain embodiments, the deuterated trehalose in the present compositions can be formulated to release up to 5, 10, 15, 20, 25, 30, 35, 40, 45, 50, 55, 60, 65, 70, 75, 80, 85, 90, 95, 99, 99.5 or 100% of the total deuterated trehalose in about 0.5, 1, 2, 3, 4, 5, 6, 7 or 8 hours. In certain embodiments, the deuterated trehalose in the present compositions can be formulated to release not less than 5, 10, 15, 20, 25, 30, 35, 40, 45, 50, 55, 60, 65, 70, 75, 80, 85, 90, 95, 99, 99.5 or 100% of the total deuterated trehalose in about 0.5, 1, 2, 3, 4, 5, 6, 7 or 8 hours.

In certain embodiments, the deuterated trehalose in the present compositions can be in a combination of sustained or slow release and immediate or fast or burst release forms. In certain embodiments, the relative proportion of sustained or slow release deuterated trehalose to immediate or fast release deuterated trehalose is, e.g., 1 to 99, 5 to 95, 10 to 90, 15 to 85, 20 to 80, 25 to 75, 30 to 70, 35 to 65, 40 to 60, 45 to 55, 50 to 50, 55 to 45, 60 to 40, 65 to 35, 70 to 30, 75 to 25, 80 to 20, 85 to 15, 90 to 10, 95 to 5, or 99 to 1.

In certain embodiments, a polymeric material is used to sustain or control release of deuterated trehalose. In certain embodiments, the type of polymeric material and the amount of which is used, have a strong influence on the rate of release of deuterated trehalose from the present compositions and delivery systems. Examples of polymers include both hydrophobic and hydrophilic polymers. Examples of hydrophobic polymers include, but are not limited to, ethyl cellulose and other cellulose derivatives, fats such as glycerol palmito-stearate, beeswax, glycowax, castorwax, carnaubawax, glycerol monostearate or stearyl alcohol, hydrophobic polyacrylamide derivatives and hydrophobic methacrylic acid derivatives, as well as mixtures of these polymers. Hydrophilic polymers include, but are not limited to, hydrophilic cellulose derivatives such as methyl cellulose, hydroxypropylmethyl cellulose, hydroxyethylcellulose, hydroxypropyl cellulose, carboxymethyl cellulose, sodium carboxymethylcellulose and hydroxyethyl methylcellulose polyvinyl alcohol, polyethylene, polypropylene, polystyrene, polyacrylamide, ethylene vinyl acetate copolymer, polyacrylate, polyurethane, polyvinylpyrrolidone, polymethylmethacrylate, polyvinyl acetate, polyhydroxyethyl methacrylate, as well as mixtures of these polymers. Furthermore, any mixture of _{\}one or more hydrophobic polymer and one or more hydrophilic polymer could optionally be used.

The deuterated trehalose contained in the present compositions and delivery systems can be entrapped in liposomes, micro- and nano-particles.

In certain embodiments, a polymeric material to be used in the present compositions and delivery systems is microcrystalline cellulose such as “Avicel PH 101” manufactured by FMC BioPolymer's. Alternatively, a polymeric material to be used in the present compositions and delivery systems is hydroxypropyl methylcellulose such as “Metholose” produced by Shin-Etsu Chemical Co. In certain embodiments, a polymeric material to be used in the present compositions and delivery systems is ethyl cellulose such as “Ethocel™” manufactured by The Dow Chemical Company. In certain embodiments, a polymeric material to be used in the present compositions and delivery systems is an acrylic polymer such as “Eudragit RS™” produced by Rohm GmbH. In certain embodiments, a polymeric material to be used in the present compositions and delivery systems is a colloidal silicone dioxide such as “Aerosil™” manufactured by Degussa. In certain embodiments, a polymeric material to be used in the present compositions and delivery systems is a Poly (Vinyl Acetate) such as “Kollicoat SR” manufactured by BASF. In certain embodiments, a polymeric material to be used in the present compositions and delivery systems is an ethyl acetate and vinyl acetate solution such as “Duro-Tak” manufactured by Delasco Dermatologic Lab & Supply, Inc.

In certain embodiments, delivery systems of the invention comprise delivery devices. In certain embodiments, the compositions of the invention are delivered by an osmotic process at a controlled rate such as by an osmotic pump. The system may be constructed by coating an osmotically active agent with a rate controlling semipermeable membrane. This membrane may contain an orifice of critical size through which agent is delivered. The dosage form after coming into contact with aqueous fluids, imbibes water at a rate determined by the fluid permeability of the membrane and osmotic pressure of the core formulation. This osmotic imbibitions of water result in formation of a saturated solution of active material with in the core, which is dispensed at controlled rate from the delivery orifice in the membrane.

In certain embodiments, the compositions of the invention are delivered using biodegradable microparticles. In certain embodiment, the system to prepare microparticles consists of an organic phase comprised of a volatile solvent with dissolved polymer and the material to be encapsulated, emulsified in an aqueous phase. In certain embodiments, the biodegradable polymers that can be used for the microparticle matrix, comprises polylactic acid (PLA) or the copolymer of lactic and glycolic acid (PLAGA). The PLAGA polymer degrades hydrolytically over time to its monomeric components, which are easily removed from the body through natural life processes.

The preparation may also contain an absorption enhancer and other optional components. Examples of absorption enhancers include, but are not limited to, are cyclodextrins, phospholipids, chitosan, DMSO, Tween, Brij, glycocholate, saponin, fusidate and energy based enhancing absorption equipment.

Optional components present in the dosage forms include, but are not limited to, diluents, binders, lubricants, surfactants, coloring agents, flavors, buffering agents, preservatives, stabilizing agents and the like.

Diluents, also termed “fillers” include, for example, dicalcium phosphate dihydrate, calcium sulfate, lactose, cellulose, kaolin, mannitol, sodium chloride, dry starch, hydrolyzed starches, silicon dioxide, colloidal silica, titanium oxide, alumina, talc, microcrystalline cellulose, and powdered sugar. For administration in liquid form, the diluents include, for example, ethanol, sorbitol, glycerol, water and the like.

Binders are used to impart cohesive qualities to the formulation. Suitable binder materials include, but are not limited to, starch (including corn starch and pregelatinzed starch), gelatin, sugars (including sucrose, glucose, dextrose, lactose and sorbitol), polyethylene glycol, waxes, natural and synthetic gums, e.g., acacia, tragacanth, sodium alginate, celluloses, and Veegum, and synthetic polymers such as polymethacrylates and polyvinylpyrrolidone.

Lubricants are used to facilitate manufacture; examples of suitable lubricants include, for example, magnesium stearate, calcium stearate, stearic acid, glyceryl behenate, and polyethylene glycol.

Surfactants may be anionic, cationic, amphoteric or nonionic surface active agents, with anionic surfactants preferred. Suitable anionic surfactants include, but are not limited to, those containing carboxylate, sulfonate and sulfate ions, associated with cations such as sodium, potassium and ammonium ions. Particularly preferred surfactants include, but are not limited to: long alkyl chain sulfonates and alkyl aryl sulfonates such as sodium dodecylbenzene sulfonate; dialkyl sodium sulfosuccinates, such as sodium bis-(2-ethylhexyl)-sulfosuccinate; and alkyl sulfates such as sodium lauryl sulfate.

Stabilizing agents such as antioxidants, include, but are not limited to, propyl gallate, sodium ascorbate, citric acid, calcium metabisulphite, hydroquinone, and 7-hydroxycoumarin.

If desired, the present compositions may also contain minor amounts of nontoxic auxiliary substances such as wetting or emulsifying agents, preservatives, and the like.

As mentioned, any of the compositions of the invention may be used alone or in combination with one or more additional therapeutic agents, for the treatment of the disease from which the subject as herein defined suffers. The amount of both the deuterated trehalose and the additional therapeutic agent that may be combined with the carrier materials to produce a composition or delivery system will vary depending upon the host treated and the particular mode of administration. In some embodiments, the compositions of this invention should be formulated so that a dosage of between 0.001-1 g/Kg body weight/day of deuterated trehalose can be administered. The dose of the deuterated trehalose depends on the condition and the illness of the patient, and the desired daily dose. In human therapy, the daily dose can be 10-3000 mg, for example, 10, 15, 20, 30, 40, 50, 75, 100, 200, 300, 400, 500, 600 700, 800, 900 or 1000 mg. These amounts are administered in doses which can be divided into 2-3 smaller doses for each day.

In certain embodiments, the active ingredients in the present compositions and delivery systems can act synergistically in combination with each other and can further act synergistically in the presence of an additional therapeutic agent. Therefore, the amount of compound(s) and additional therapeutic agent(s) in such compositions will be less than that required in a monotherapy utilizing only that therapeutic agent.

Toxicity and therapeutic efficacy of the formulations described herein can be determined by standard pharmaceutical procedures in cell cultures or experimental animals, e.g., procedures used for determining the maximum tolerated dose (MID), the ED₅₀, which is the effective dose to achieve 50% of maximal response, and the therapeutic index (TI), which is the ratio of the MTD to the ED₅₀. Obviously, formulations with high TIs are the most preferred formulations herein, and preferred dosage regimens are those that maintain plasma levels of the deuterated trehalose at or above a minimum concentration to maintain the desired therapeutic effect. Dosage will, of course, also depend on a number of factors, the site of intended delivery, the route of administration, and other pertinent factors known to the prescribing physician.

As indicated above, deuterated trehalose or any of the formulations comprising thereof as herein defined may be used alone or in combination with one or more additional therapeutic agents for the treatment of the diseases from which the treated subjects suffer. The amount of both the deuterated trehalose and the additional therapeutic agent that may be combined with deuterated trehalose or any of the formulations comprising thereof as herein defined will vary upon the subject treated and the particular diseases and mode of administration.

In yet another embodiment the presently disclosed subject matter provides an aqueous pharmaceutical formulation for parenteral administration, comprising a therapeutically effective amount of deuterated trehalose as a sole active ingredient, wherein the formulation has a pH about 4.5 to 7.0 and contains less than 0.74 endotoxin units per ml and wherein in said deuterated trehalose at least one hydrogen atom attached to a carbon atom is replaced by a deuterium atom.

In certain embodiments aqueous pharmaceutical formulations according to the present disclosure is wherein said deuterated trehalose is α,α-deuterated trehalose.

In further embodiments in the aqueous pharmaceutical formulations according to the present disclosure said deuterated trehalose is selected from α,α-[1,1′-²H₂]trehalose having a structure according to Formula I

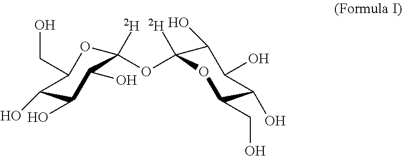

α,β-[1,1′-²H₂]trehalose having a structure according to Formula II

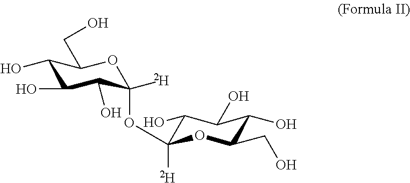

β,β-[1,1′-²H₂]trehalose having a structure according to formula III

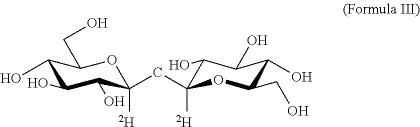

α,α-[UL-²H₁₄]trehalose having a structure according to formula IV

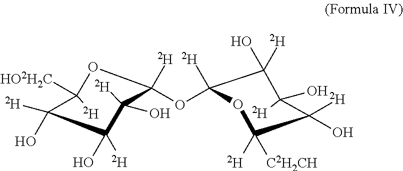

α,β-[UL-²H₁₄]trehalose, having a structure according to formula V

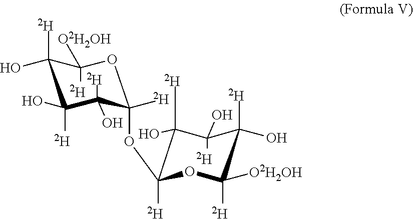

β,β-[UL-²H₁₄]trehalose having a structure according to formula VI

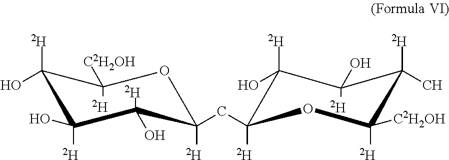

a deuterated trehalose molecule having a structure according to Formula VII

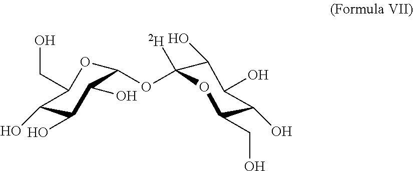

a deuterated trehalose having a structure according to Formula VIII

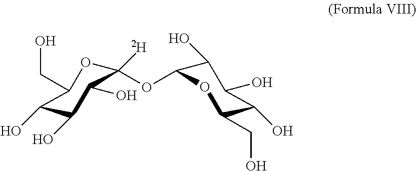

a deuterated trehalose having a structure according to Formula IX

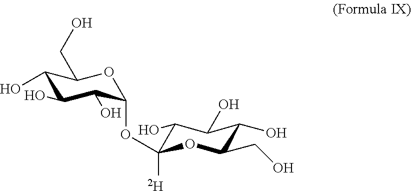

a deuterated trehalose having a structure according to Formula X

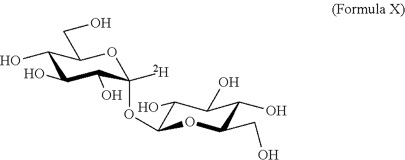

a deuterated trehalose having a structure according to Formula XI

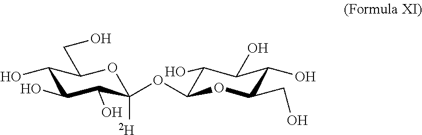

and a deuterated trehalose having a structure according to Formula XII

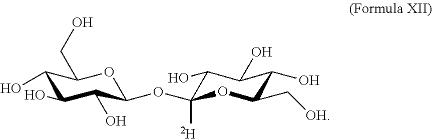

In some embodiments said aqueous pharmaceutical formulation as herein defined is adapted for parenteral or enteral administration. Specific aqueous pharmaceutical formulations for parenteral administration as herein defined may be adapted for intravenous, intramuscular or intraperitoneal administration. Specific aqueous pharmaceutical formulations for enteral administration as herein defined may be adapted for oral or intragastric administration.

In some embodiments the aqueous pharmaceutical formulation as herein defined optionally further comprises at least one pharmaceutically acceptable additive, carrier, excipient or diluent.

In further embodiments in the aqueous pharmaceutical formulations as herein defined the concentration of the deuterated trehalose is between about 0.1% (w/v) to about 50% (w/v). In further specific embodiments the aqueous pharmaceutical formulation as herein defined is wherein the concentration of the deuterated trehalose is about 10% (w/v). In some embodiments aqueous pharmaceutical formulations as herein defined, particularly when designed for parenteral administration, have an osmolality of about 280-330 mOsm/kg.

In other embodiments the aqueous pharmaceutical formulations as herein defined are administered at a frequency between once daily to once per month.

In aqueous pharmaceutical formulations as herein defined the therapeutically effective amount of deuterated trehalose is from about 1 gram to about 100 gram for each daily administration and no more than about 1 gram/Kg body weight, for example no more than 0.25 gram/Kg body weight or 0.5 gram/Kg body weight of said subject per day.

In other embodiments aqueous pharmaceutical formulations as herein defined can be administered once daily at from about 1 mg/kg/day to about 1 gram/kg/day of deuterated trehalose.

In still further embodiments aqueous pharmaceutical formulations as herein can be administered at a frequency of between once daily to once per month at a dose of about 5 to about 35 grams of deuterated trehalose.

In further specific embodiments aqueous pharmaceutical formulations as herein defined are designed for administration once daily, once every other day, twice a week, once a week, once every two weeks, once every three weeks or once a month.

In still further specific embodiments aqueous pharmaceutical formulations as herein defined can be administered at a dose of 5, 8, 15, 30, 40 or 50 grams.

In yet further embodiments, aqueous pharmaceutical formulations as herein can be administered at a rate of administration such that the maximum endotoxin level is less than 5 endotoxin units per kilogram of body weight of the patient per hour.

Aqueous pharmaceutical formulations of the presently disclosed subject matter can be adapted for intravenous administration wherein the administration is completed within from about 75 to about 120 minutes, specifically within less than 90 minutes.

The presently disclosed subject matter further provides a solid dosage forms for oral administration, comprising deuterated trehalose as active ingredient, and further optionally comprising at least one of carrier, additive or excipient.

In certain embodiments the solid dosage form of the present disclosure is wherein said deuterated trehalose is α,α-deuterated trehalose.

In some embodiments, the aqueous pharmaceutical formulation or the solid dosage as herein defined are designed for treating a disease associated with abnormal protein aggregation and/or inclusion bodies formation in neurons, myocytes and other cells or extracellular compartments or for alleviating a sign or symptom associated therewith in a human subject in need thereof.

In specific embodiments the aqueous pharmaceutical formulation or the solid dosage form as herein defined is wherein said disease is any one of Huntington's disease (HD), oculopharengeal muscular dystrophy (OPMD), spinocerebellar ataxia (SCA), Friedreich's ataxia, spinal and bulbar muscular atrophy (SBMA), Parkinson's disease, Alzheimer's disease, amyotrophic lateral sclerosis (ALS), dentatombral-pallidoluysian atrophy (DRPLA), Pick's disease, Corticobasal degeneration (CBD), Progressive supranuclear palsy (PSP) and Frontotemporal dementia and parkinsonism linked to chromosome 17 (FTDP-17).

The presently disclosed subject matter further provides a kit comprising:

(a) pharmaceutically acceptable deuterated trehalose or active derivative thereof;
(b) at least one pharmaceutically acceptable additive, carrier, excipient and diluent;
(c) means for preparing an injectable aqueous solution of the deuterated trehalose by mixing said deuterated trehalose with at least one of said additive, carrier, excipient and diluent;
(d) means for parenterally administering said injectable solution to a patient in need;
(d) instructions for use.

The presently disclosed subject matter further provides a kit comprising:

(a) pharmaceutically acceptable deuterated trehalose or active derivative thereof;
(b) at least one pharmaceutically acceptable additive, carrier and excipient and fro producing a solid, enterally administrable dosage form;
(c) means for mixing said deuterated trehalose with said at least one of said additive, carrier and excipient to form a solid dosage form;
(d) instructions for use.

Injectable aqueous solutions of the deuterated trehalose can be prepared by any method well known in the art, for example as recited herein in the accompanying Examples. Parenterally administering the injectable solution as herein defined is well known in the art of the skilled physician.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

The invention will be further described in the following examples, which do not limit the scope of the invention described in the claims.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook & Russell, 2001.

Standard medicinal chemistry methods known in the art not specifically described herein are generally followed essentially in the series “Comprehensive Medicinal Chemistry” by various authors and editors, published by Pergamon Press.

### Example 1

### Alpha-Alpha Deuterated Trehalose

Alpha-alpha deuterated trehalose (α-α deuterated trehalose) is obtained from commercial sources. Samples of the compound are kept at room temperature until analysis, and are analyzed by accepted methods to verify their specifics, in order to verify that the tested α-α deuterated trehalose preparations conform with the accepted practice concerning use of trehalose in pharmaceutical compositions, namely that they comprise 97.0-102.0% w/w active ingredient (α-α deuterated trehalose), with any peak eluting before α-α deuterated trehalose at a concentration of less than 0.5% w/w, glucose at less than 0.5% w/w and with any peak eluting after α-α deuterated trehalose at less than 0.5% w/w.

### Example 2

### Preparation of α-α Deuterated Trehalose Solution for IV Injection

A formulation comprising α-α deuterated trehalose is prepared under sterile conditions by dissolving α-α deuterated trehalose in water (and optionally supplemented with additional excipients and/or carriers) and the resulting liquid is analyzed using HPLC to identify any impurities or contaminants, for example glucose, maltotriose and other polysaccharides. In addition, the pH, osmolality, endotoxin content and sterility of the formulation is analyzed.

### Example 3

### Preclinical Pharmacokinetic Studies

The plasma, nerve and muscle concentrations of α-α deuterated trehalose in male Sprague-Dawley (SD) rats is determined after intravenous bolus (IV) and oral gavage (PO) administration.

All applicable portions of the study confirm to the following regulations and guidelines regarding animal care and welfare: AAALAC International and NIH guidelines as reported in the “Guide for the Care and Use of Laboratory Animals,” National Research Council ILAR, Revised 1996.

The study includes 42 SD rats (male, 250 to 350 grams in weight, the Shanghai SLAC Laboratory Animal Co. Ltd.). Animals are administered with deuterated trehalose formulation (α-α deuterated trehalose in sterilized water at 200 mg/mL).

Blood samples are collected after each dose administration and processed for plasma. Muscle and nerve samples are collected and homogenized. The concentrations of trehalose in plasma, muscle and nerve homogenate samples are analyzed by qualified bioanalytical LC/MS/MS methods.

### Pharmacokinetics Data Analysis

Plasma concentration data of α-α deuterated trehalose is subjected to a non compartmental pharmacokinetic analysis using WinNonlin software program (version 6.3, Pharsight, Mountain View, Calif.). Zero-time intercept concentration (CO), volume of distribution (Vdss), Clearance (Cl), peak plasma concentrations (Cmax) and the corresponding peak times (Tmax), terminal half-life (T1/2), mean residence time (MRT) from time zero to the last time point (MRT0−last), MRT from time zero to infinity (MRT0−inf), the area under the plasma concentration-time curve (AUC) from time zero to the last time point (AUC0−last) and AUC from time zero extrapolated to infinity (AUC0−inf) are calculated using the linear/log trapezoidal rule. Nominal sampling times are used to calculate all pharmacokinetic parameters in case there is no deviation larger than 5% between the actual and nominal sampling times.

The values of muscle to plasma and nerve to plasma concentration and AUC ratio (M/P ratio) are calculated as well.

### Trehalose Concentration in Plasma, Muscle and Nerve

Pharmacokinetic parameters of α-α deuterated trehalose in the plasma, muscle and nerve are examined following single intravenous or oral administration of α-α deuterated trehalose solution (200 mg trehalose dihydrate per 1 mL sterilized water) at various doses to male SD rats. The pharmacokinetic parameters of α-α deuterated trehalose in the plasma, muscle and nerve are compared to the pharmacokinetic parameters of trehalose under the same assay conditions.

### Example 4

### Determination of Endotoxin Level

It is accepted that the maximal allowed level of endotoxin in formulations administered intravenously is 5 endotoxin units (EU) per kg body mass per hour (5 EU/kg/hr). In order to determine the theoretical maximum endotoxin level IV per kg body mass/hour (K) in α-α deuterated trehalose formulation (solution of α-α deuterated trehalose dihydrate in sterilized water), the maximal endotoxin levels in α-α deuterated trehalose formulations are calculated based on the contribution of the endotoxin from the α-α deuterated trehalose, the solvent used and the rate at which the formulation is being administered intravenously.

### Example 5

### Safety Studies

Trehalose is known to be safe and tolerable. For example, non-deuterated trehalose is recognized as a safe food ingredient as well as a GRAS material used in the pharmaceutical industry as an excipient for oral, intraocular and IV drug formulations. In several studies, healthy volunteers were given oral doses of trehalose ranging from 10 to 60 gr. Apart from mild abdominal symptoms (e.g. flatulence, distension, borborygmus and occasional diarrhea) no other safety issues were reported [18]. Further, non-deuterated trehalose has been used as a protein stabilizer in several commercially available protein drugs for over a decade and its safety has repeatedly been established in patient populations at advanced stages of malignant diseases, hemophilia and related clotting disorders. These drugs are approved for use for several years, and are sometimes given to patients as frequently as every 8 hours through 2-3 weeks intervals.

The safety and tolerability of α-α deuterated trehalose are investigated, as detailed below. α-α deuterated trehalose median lethal dose (LD₅₀) is examined in mice, rats and dogs.

### Example 6

### Treatment of R6/1 and R6/2 Transgenic Mice with α-α Deuterated Trehalose

It has been previously shown that various disaccharides reduced polyglutamine aggregates and increased survival in a cellular model of Huntington disease [7]. As indicated above, it was also reported that oral administration of trehalose, the most effective of these disaccharides, decreased polyglutamine aggregates in cerebrum and liver, improved motor dysfunction and extended lifespan in a transgenic mouse model of Huntington disease.

It has been suggested that these beneficial effects are the result of trehalose binding to expanded polyglutamines and stabilizing the partially unfolded polyglutamine-containing protein.

The effect of α-α deuterated trehalose on Huntington disease is tested in R6/1 and R6/2 transgenic mice, that express exon 1 of the human HD gene with around 115 and 150 CAG repeats, respectively. The R6/1 and R6/2 transgenic mice were the first transgenic mouse models developed to study HD [18]. The transgene expression in these mice is driven by the human huntingtin promoter. The resulting levels of transgene expression are around 31% and 75% of the endogenous huntingtin in the R6/1 and R6/2 models, respectively.

In order to examine the effect of α-α deuterated trehalose on Huntington disease, R6/1 and R6/2 transgenic mice are administered with α-α deuterated trehalose, both orally and intravenously, from the age of 21 days until they are sacrificed. The levels of α-α deuterated trehalose in the plasma and in the nerve, brain and muscle tissues are determined. The influence of the α-α deuterated trehalose on brain atrophy is examined. The effects of α-α deuterated trehalose on formation of polyglutamine aggregates in motor cortex, striatum and liver is examined by immunohistochemistry. Further, the effects of α-α deuterated trehalose on the motor function of 7-11 weeks old treated mice are assessed by rotarod and foot-printing tests. Other assessments are also conducted, for example the effect of α-α deuterated trehalose on foot clasping posture. In addition, the effect of α-α deuterated trehalose on survival and possible lifespan extension is also investigated

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating or alleviating a disease associated with abnormal protein aggregation and/or inclusion bodies formation in myocytes, neurons and other cells or extracellular compartments or at least one symptom associated therewith, in a human subject in need thereof comprising administering to said subject a therapeutically effective amount of deuterated trehalose or a mixture of several deuterated trehaloses, optionally together with non-deuterated trehalose or a pharmaceutical formulation comprising a therapeutically effective amount of deuterated trehalose or a mixture of several deuterated trehaloses, and optionally comprising non-deuterated trehalose, wherein in said deuterated trehalose at least one hydrogen atom attached to a carbon atom is replaced by a deuterium atom.

2. The method of claim 1, wherein said deuterated trehalose is α,α-deuterated trehalose.

3. The method of claim 1, wherein said deuterated trehalose is selected from α,α-[1,1′-²H₂]trehalose having a structure according to Formula I 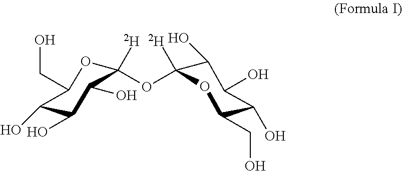
α,β-[1,1′-²H₂]trehalose having a structure according to Formula II 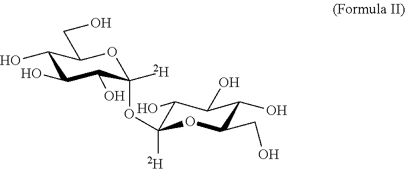
β,β-[1,1′-²H₂]trehalose having a structure according to formula III 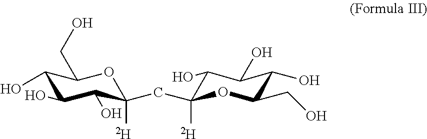
α,α-[UL-²H₁₄]trehalose having a structure according to formula IV 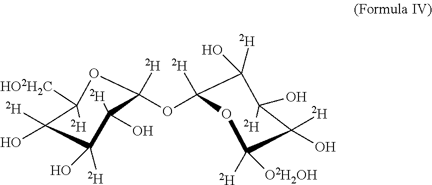
α,β-[UL-²H₁₄]trehalose, having a structure according to formula V 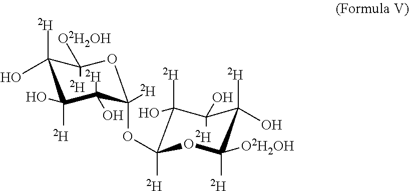
β,β-[UL-²H₁₄]trehalose having a structure according to formula VI 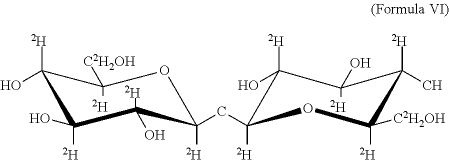
a deuterated trehalose molecule having a structure according to Formula VII 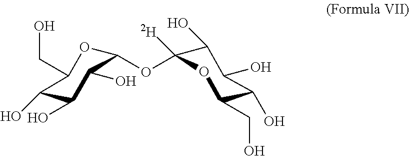
a deuterated trehalose having a structure according to Formula VIII 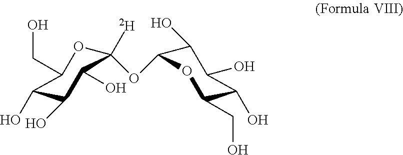
a deuterated trehalose having a structure according to Formula IX 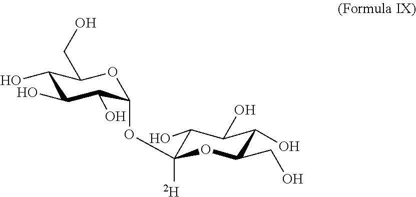
a deuterated trehalose having a structure according to Formula X 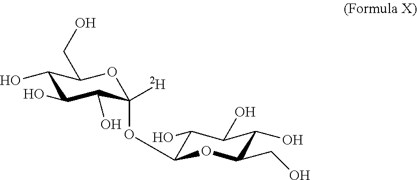
a deuterated trehalose having a structure according to Formula XI 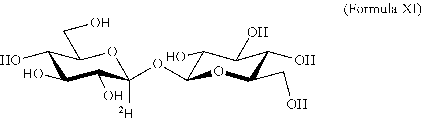
and a deuterated trehalose having a structure according to Formula XII

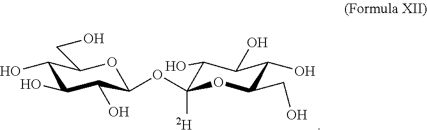

4. The method of claim 1, wherein said disease is any one of a neurodegenerative disorder, poly-alanine aggregation disorder, poly-glutamine aggregation disorder, a protein codon reiteration disorder, a myopathy and a tauopathy.

5. The method of claim 1, wherein said disease is any one of Huntington's disease, oculopharengeal muscular dystrophy (OPMD), spinocerebellar ataxias (SCA), Friedreich's ataxia, spinal and bulbar muscular atrophy (SBMA), Parkinson's disease, Alzheimer's disease and amyotrophic lateral sclerosis (ALS), dentatorubral-pallidoluysian atrophy (DRPLA), Pick's disease, Corticobasal degeneration (CBD), Progressive supranuclear palsy (PSP) and Frontotemporal dementia and parkinsonism linked to chromosome 17 (FTDP-17).

6. The method of claim 1, wherein said deuterated trehalose or a pharmaceutical formulation comprising thereof is administered parenterally.

7. The method of claim 1, wherein said pharmaceutical formulation is an injectable solution for parenteral administration.

8. The method of claim 1, wherein said deuterated trehalose or said mixture of several deuterated trehaloses, optionally together with non-deuterated trehalose, or a pharmaceutical formulation comprising thereof is administered enterally, specifically by oral administration.

9. The method of claim 8, wherein said pharmaceutical formulation is an aqueous solution.

10. The method of claim 8, wherein said pharmaceutical formulation is a solid dosage form.

11. The method of claim 10, wherein said pharmaceutical formulation comprises deuterated trehalose or a mixture of several deuterated trehaloses, optionally together with non-deuterated trehalose, as sole active ingredient, and optionally further comprises at least one pharmaceutically acceptable additive, carrier, excipient or diluent.

12. The method of claim 11, wherein the concentration of deuterated trehalose or a mixture of several deuterated trehaloses, optionally together with non-deuterated trehalose, in said formulation is between about 0.1% (w/v) to about 50% (w/v).

13-14. (canceled)

15. The method of claim 13, wherein said parenteral administration is any one of intravenous, intramuscular and intraperitoneal administration.

16-19. (canceled)

20. The method of claim 1, wherein said therapeutically effective amount of deuterated trehalose or mixture of several deuterated trehaloses, optionally together with non-deuterated trehalose, or pharmaceutical formulation comprising thereof is administered at a frequency of between once daily to once per month.

21. The method of claim 20, wherein said therapeutically effective amount of deuterated trehalose or mixture of several deuterated trehaloses, optionally together with non-deuterated trehalose, or deuterated trehalose or mixture of several deuterated trehaloses, optionally together with non-deuterated trehalose, comprised in said pharmaceutical formulation is administered once daily at from about 1 mg/kg/day to about 1 gram/kg/day of deuterated trehalose.

22. The method of claim 21, wherein said therapeutically effective amount of deuterated trehalose or mixture of several deuterated trehaloses, optionally together with non-deuterated trehalose, or deuterated trehalose comprised or mixture of several deuterated trehaloses, optionally together with non-deuterated trehalose, in said pharmaceutical formulation is administered at a single injection administration.

23-25. (canceled)

26. The method of claim 1, wherein administration of said therapeutically effective amount of deuterated trehalose or mixture of several deuterated trehaloses, optionally together with non-deuterated trehalose, comprised in said pharmaceutical formulation adapted for intravenous administration is completed within from about 75 to about 120 minutes, specifically within less than 90 minutes.

27.-29. (canceled)

30. An aqueous pharmaceutical formulation for any one of enteral or parenteral administration, comprising a therapeutically effective amount of deuterated trehalose or mixture of several deuterated trehaloses, optionally together with non-deuterated trehalose, as a sole active ingredient, wherein in any of said deuterated trehalose at least one hydrogen atom attached to a carbon atom is replaced by a deuterium atom, optionally further comprising at least one of pharmaceutically acceptable additive, excipient, diluent and carrier.

31. (canceled)

32. The aqueous pharmaceutical formulation according to claim 30, wherein said deuterated trehalose is selected from α,α-[1,1′-²H₂]trehalose having a structure according to Formula I 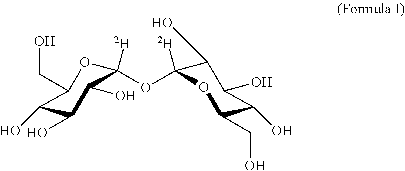
α,β-[1,1′-²H₂]trehalose having a structure according to Formula II 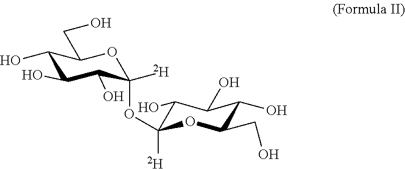
β,β-[1,1′-²H₂]trehalose having a structure according to formula III 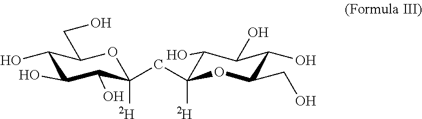
α,α-[UL-²H₁₄]trehalose having a structure according to formula IV 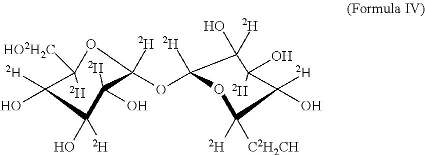
α,β-[UL-²H₁₄]trehalose, having a structure according to formula V 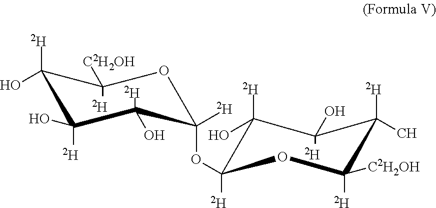
β,β-[UL-²H₁₄]trehalose having a structure according to formula VI 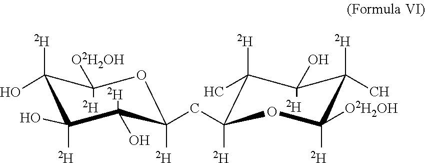
a deuterated trehalose molecule having a structure according to Formula VII 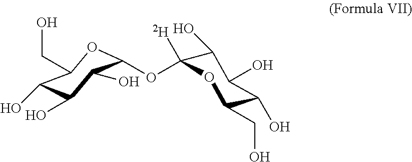
a deuterated trehalose having a structure according to Formula VIII 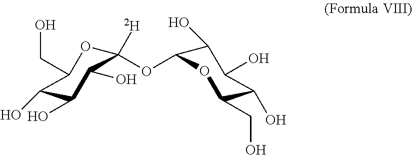
a deuterated trehalose having a structure according to Formula IX 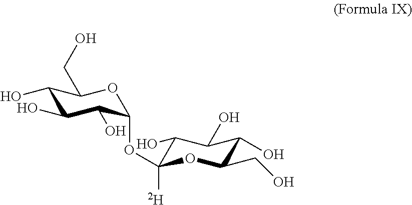
a deuterated trehalose having a structure according to Formula X 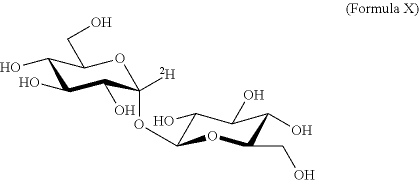
a deuterated trehalose having a structure according to Formula XI 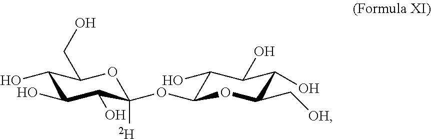
and
a deuterated trehalose having a structure according to Formula XII

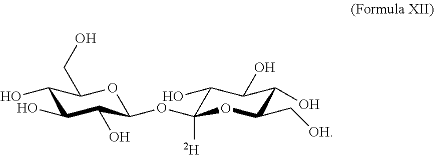

33-52. (canceled)

53. A kit comprising:
(a) pharmaceutically acceptable deuterated trehalose or active derivative thereof;
(b) at least one pharmaceutically acceptable additive, carrier, excipient and diluent;
(c) means for preparing an injectable aqueous solution of the deuterated trehalose by mixing said deuterated trehalose with at least one of said additive, carrier, excipient and diluent;
(d) means for parenterally administering said injectable solution to a patient in need;
(e) instructions for use.
